SEQUENCE LISTING (53 sequences)

SEQ ID NO: 1 - DNA, 2893 bp, Homo sapiens
ggcacgaggatcaggaagggggtgcaagagggttagtgattggggagcagaaggggtcctaaagatcgctctgggaaaagggaaggatgccgctcttcttccggaagcggaaacccagtgaggaggctcggaaacgcctggagtaccagatgtgtttggcaaaagaagctggggcagatgacattctcgacatctctaaatgtgagctctcagagattccatttggagcttttgcaacatgcaaagttctgcagaagaaggtgctgatcgtccacacgaatcacctcacttccctgcttcccaaatcctgcagcctcctgagtctggcaaccattaaggttctagatctccacgataatcagctgacagcccttcctgacgatctggggcagctgactgccctccaggtcttaaacgtggaaaggaatcaactgatgcagctcccacgttccattgggaacctgacccagctccagactctcaatgttaaagacaacaagctgaaggagcttccagacaccgtgggggagcttcgaagcctgcgtaccctcaacatcagtggaaacgagatccagagattgccgcagatgctggctcacgttcgaaccctggagatgctgagccttgacgcctcggccatggtctacccgccgcgggaggtgtgtggtgccggcactgcggccatcttgcagttcctctgcaaagagtcagggctggaatactaccccccttctcagtacttgctgccaattctggagcaagatggaatcgagaactctcgggacagccctgatgggcccacggacagattctcaagggaggagttagagtggcagaacaggttctcagactatgagaagaggaaggaacagaagatgctggagaaactcgagtttgaacggcgcctggaactggggcagcgggagcacacccagctccttcagcagagcagcagccagaaggatgagatccttcagacggtcaaggaggagcagtcccggctggagcagggcctgagtgagcaccagcgccacctcgacgcagagcggcagcggctgcaggagcagctgaagcagacggaacagaacatttccagccggatccagaagctgctgcaggacaatcagagacaaaagaaaagctccgagattttgaaatcgctggaaaatgaaagaataagaatggaacagttgatgtccataacccaggaggagactgagagcctgcggcgacgtgacgttgcctccgccatgcagcagatgctgactgagagctgtaagaaccggctcatccagatggcctacgaatctcagaggcagaacttggtccagcaggcctgttccagcatggccgaaatggatgaacgattccagcagattctgtcgtggcagcaaatggatcagaacaaagccatcagccagatcctgcaggagagcgcgatgcagaaggctgcgttcgaggcactccaggtgaagaaagacctgatgcatcggcagatcaggagccagattaagttaatagaaactgagttattgcagctgacacagctggagttaaagaggaagtccctggacacagagtcactccaggagatgatctcggagcagcgctgggccctcagctccctgctccagcagctgctcaaagagaagcagcagcgagaggaagagctccgggaaatcctgacggagttagaagccaaaagtgaaaccaggcaggaaaattactggctgattcagtatcaacggcttttgaaccagaagcccttgtccttgaagctgcaagaagaggggatggagcgccagctggtggccctcctggaggagctgtcggctgagcactacctgcccatctttgcgcaccaccgcctctcactggacctgctgagccaaatgagcccaggggacctggccaaggtgggcgtctcagaagctggcctgcagcacgagatcctccggagagtccaggaactgctggatgcagccaggatccagccagagctgaaaccaccaatgggtgaggtcgtcacccctacggccccccaggagcctcctgagtctgtgaggccatccgctccccctgcagagctggaggtgcaggcctcagagtgtgtcgtgtgcctggaacgggaggcccagatgatcttcctcaactgtggccacgtctgctgctgccagcagtgctgccagccactgcgcacctgcccgctgtgccgccaggacatcgcccagcgcctccgcatctaccacagcagctgagtgctgcccgcccacctgggcctggtcctagccctgcctcggccactgtgagccccgggctcctgctcagccttgtgccagccagactcgtatgaggctcccccctgccctgggccccttccccactgcccaggagcccccatcctaagctccaagcatgtctgggccaggcagaggtgctcctcatccatgacaccaccagtctgaatggtcctgggggctggggctggagaggccgctgcaccaccacccgagcctgggagccagcgtcccagcctaatcacggatctgctgcctcccagctgtcttgactgaaggccaccgcccctgcaggagcttgggtcctcatctgggggccatgcacaggcccgtcccaccctgcatgtgggaagggagcaggagggcctggctgggtgaggggaggccttcctgggaaggcgtgtggtgcaggcctgtgctcacagtggcaccagcaaccctgggtctccctctctgctgctccccagaaccccggggccctcctgctctccacaactgtccctccttaccccatgtagctcgatccgaagcaggagtgtcaataaacctgtcttcagtgcgaaaaaaaaaaaaaaaaaaaaaaaaaaaaaaaaaaa

SEQ ID NO: 2 - protein, 723 aa, Homo sapiens
MPLFFRKRKPSEEARKRLEYQMCLAKEAGADDILDISKCELSEIPFGAFATCKVLQKKVLIVHTNHLTSLLPKSCSLLSLATIKVLDLHDNQLTALPDDLGQLTALQVLNVERNQLMQLPRSIGNLTQLQTLNVKDNKLKELPDTVGELRSLRTLNISGNEIQRLPQMLAHVRTLEMLSLDASAMVYPPREVCGAGTAAILQFLCKESGLEYYPPSQYLLPILEQDGIENSRDSPDGPTDRFSREELEWQNRFSDYEKRKEQKMLEKLEFERRLELGQREHTQLLQQSSSQKDEILQTVKEEQSRLEQGLSEHQRHLDAERQRLQEQLKQTEQNISSRIQKLLQDNQRQKKSSEILKSLENERIRMEQLMSITQEETESLRRRDVASAMQQMLTESCKNRLIQMAYESQRQNLVQQACSSMAEMDERFQQILSWQQMDQNKAISQILQESAMQKAAFEALQVKKDLMHRQIRSQIKLIETELLQLTQLELKRKSLDTESLQEMISEQRWALSSLLQQLLKEKQQREEELREILTELEAKSETRQENYWLIQYQRLLNQKPLSLKLQEEGMERQLVALLEELSAEHYLPIFAHHRLSLDLLSQMSPGDLAKVGVSEAGLQHEILRRVQELLDAARIQPELKPPMGEVVTPTAPQEPPESVRPSAPPAELEVQASECVVCLEREAQMIFLNCGHVCCCQQCCQPLRTCPLCRQDIAQRLRIYHSS

SEQ ID NO: 3 - DNA, 2044 bp, Mus musculus
cttggtttctagaatctcgagactttgtcatcctgagttgcgtgtctttctgaaatttaaagtttcggtgctcacttctatgtttgaaggagaccggacaccagctcagcttttgggggccaatggtttgtatctgtggccaagtcttcggagtgactggcctaccttgaggtccacccaagaatcggaacatcggtggaggacctccccatccacagagccagggtccagaagagctcacaccggaggatgcccctcttctttcggaagcggaaacccagtgaggaggctcgaaaacgcctggagtaccagatgtgtctggcaaaagaagctggggcagatgacattctcgacatctctaaatgtgagctctctgagattccatttggggcttttgcaacgtgcaaagttctacagaaaaaggtgttgattgtccatacaaaccacctcacctccctgcttcccaagtcctgcagcctcttgagccttgtcaccatcaaggttctggatctccatgagaaccagctgacagcccttcctgatgacatggggcagctgacagtcctgcaggtattgaatgtggaaagaaatcaactcacgcatctccctcgctctattgggaacctgctgcagctccagacgctcaatgtaaaagacaacaagctgaaggagcttcctgacaccctgggggagctgcggagcctgcggacactcgacattagtgagaacgagattcagagacttccccagatgctggcgcacgtgcggaccctggagacgctgagcctcaacgccttggcaatggtctaccccccaccagaggtgtgtggcgctggcactgcggccgtgcagcagttcctctgcaaagagtcaggactggactattacccaccttctcagtacctgctgccagtcctggagcaagatggagcagagaacacccaagacagccccgatggacccgcaagccgattctccagggaggaggctgaatggcagaatcggttctccgactacgagaagcggaaggagcagaagatgctggagaagctggagttcgagcggcgcctggaccttgggcagcgggagcacgctgagctactgcagcagagccacagccacaaggacgagatcctgcagacggtcaagcaggagcagacacggctagagcaggacctgagcgagcgccagcgctgtctggatgcagagcggcagcagctgcaggagcagctcaagcagacggagcagagcatcgccagccgcattcagagactcctgcaggacaaccagaggcaaaagaagagttctgagattctgaaatcgctggagaatgagagaataagaatggagcagttgatgtccatcacccaggaggagacagagaacctcaggcagcgtgagatcgccgccgccatgcagcagatgctgacggagagctgtaagagccggctcatccagatggcctatgagtctcagaggcagagcctggcgcagcaggcctgttccagcatggctgaaatggacaagcggttccagcagattctgtcttggcagcagatggatcagaacaaagccatcagccagatccttcaggagagtgtaatgcagaaggctgccttcgaggctctccaggtgaagaaggacctgatgcatcggcagatcaggaaccagattaggctaatagaaactgagttactgcagctgacacagctggagttaaagaggaagtccctggacacagagacgcttcaggagatggtctcagagcagcgctgggcactcagcaacctgctccagcagctcctgaaagagaagaagcagcgggaagaggaactccatggcatcctggcggaattagaggccaagagcgaaacgaagcaggaaaattactggctcatccagtaccaacggcttttaaaccagaagcctttgtccttgaaactgcaggaagaaggcatggagcgacggctggtggccctgctggtggagctttctgcagagcactacctgcccctcttcgcccaccaccgcatctcactggaca

SEQ ID NO: 4 - protein, 116 aa, Mus musculus
MFEGDRTPAQLLGANGLYLWPSLRSDWPTLRSTQESEHRWRTSPSTEPGSRRAHTGGCPSSFGSGNPVRRLENAWSTRCVWQKKLGQMTFSTSLNVSSLRFHLGLLQRAKFYRKRC

SEQ ID NO: 5 - DNA, 2971 bp, Rattus norvegicus
ggtccagaagaactctcgcaggaggatgcctctcttctttcggaagcggaaacccagtgaggaagctcggaaacgcctggagtaccagatgtgtctggcaaaagaagctggggcagatgacatccttgacatctctaagtgcgagctttccgagattccatttggggcttttgcaacgtgcaaagttctacagaaaaaggtgttgattgtccacacaaaccatctcacctccctgctgcccaagtcctgcagcctcttgagcctcgccaccatcaaggttctggatctccatgacaaccagctgacagcccttcctgacgatattgggcagctgacagccctgcaggtattgaatgtagaaaggaatcaactgacacacctcccacgctctgttgggaacctgctgcagctccagaccctcaacgtaaaaggtggggacacaagccctgtgcacgttaccctcaggcaactccagagtcaggccaccgagtgtgagggtgacggatcagtctgtctccatggcaaccagaagcagtatgtctatgagcccgagagtcagagacttgtggggcagaagacagacagacagaccatcacagtgacagaacgagacaacaagctaaaggagcttccggacaccctgggggagctgcggagcctgcgtaccctcgacatcagtgaaaatgagatccagagacttccccagatgctggctcatgtgcggaccctggagatggttctgaacaaccctgtggctgtcacctctgcaaagcttagtatttgtcacagtggtaacaacctggccgagcatcccagtccccgctccccctgcttttgtgaatcacccctgtcaagccagactgaggagcagcagtgtctggggaagtggcagacgctgagcctcgatgccttgtcaatggtctaccccccaccagaggtgtgtggcgctggcactgcggccgtgcagcagttcctctgcaaagagtcaggcctggactattacccaccttctcagtacctgctgccagtcctggagcaagatggagccgagaactcccaggacagccctgatggacccacacgcagattctccagggaggaggctgaatggcagaatcggttctccgactacgagaagcgaaaggagcagaagatgctggagaagctggagttcgagcggcgcctggacctcgggcagcgggagcatgctgagctgctccagcagagccacagccacaaggacgagatcctgcagacggtcaagcaggagcagacacggctcgagcagggcctgagtgagcgccagcgctgcctggatgcagaacggcagcagctgcaggagcagctcaagcagtcggagcagagcattgccagccgcatccagagactcctgcaggacaatcagaggcaaaagaagagttctgagattctgaaatcactggagaatgagagaatacgaatggagcagctgatgtccattacccaggaggagaccgagaacctcaggcagcgtgagatcgccgccgccatgcagcagatgctgaccgagagctgtaagagccggctcatccagatggcctatgagtcccagaggcagagcctggtgcagcaggcctgttccagcatggctgaaatggacaagcggttccagcagattctgtcatggcagcagatggaccagaacaaagccatcagccagatccttcaggaggctcgaatgctgcttgcagttgattacaaacacgcgatgtgtccagtcctgtctttgctgaaggctgtttcttacaggcaacagcagctgaatcccatccattttcgtttagatgtggagttgaggacccaggactggaggcccctctttgtccttctgtccctggtgtttggggctgtcctcgtcccacctgtggtttcgggtgctcttctccgtcttcagaatgccagtcacctggctgtttgcagtcagcgtcatgtggatgtgtcagatgagcgtctgacctcagaacctccgttgttcatcctcagtgtgatgcagaaggctgcattcgaggctctccaggtaaagaaagacctcacgcatcggcagatcaggagccagattaggctaatagaaactgagttactgcagctgacacagctggagttaaagaggaagtccctggacacagagacgcttcagggcggctgctcctcagctccagacacaggcttctccggcacacagagagccggcccagccccagtagaacagatgtggtccatgggcaaaggtagctctgtgcagggcgagagggagatggtctcagagcagcgctgggcgctcagcaacctgctccagcagctcctcaaagagaagaagcagcgggaagaggagctccatggcatcctggcggaattagaggccaagagtgaaacgaagcaggaaaattactggctcatccagtaccaacggcttttgaaccagaagcctttgtccttgaagctgcaggaagaaggcatggagcggcagctggtggccctgctggtggagctgtctgctgagcactacctgcccctcttcgcccaccaccgcatcacactggacatgctgagccggatgggtcctggagatctggctaaggtgggtgtctcagaagcaggcctgcaacatgaaatcctgcgaagagcccgggacctgctggatgtggccagggtccaaccagagttgaaaccacccaagaatgaggtctttggtgtctctgagccccccacagcccctcaggagcttcctgagtccgtgagaccatctgccccgccagctgaactggacgtgccgacctcagagtgtgttgtgtgcctggaacgtgaggcccagatggtcttcctcacctgcggccatgtctgctgctgccagcagtgctgccagccgctgcgcacctgcccactgtgccgccaggagatctcccagcgcctccggatctaccacagcagctga

SEQ ID NO: 6 - protein, 981 aa, Rattus norvegicus
MPLFFRKRKPSEEARKRLEYQMCLAKEAGADDILDISKCELSEIPFGAFATCKVLQKKVLIVHTNHLTSLLPKSCSLLSLATIKVLDLHDNQLTALPDDIGQLTALQVLNVERNQLTHLPRSVGNLLQLQTLNVKGGDTSPVHVTLRQLQSQATECEGDGSVCLHGNQKQYVYEPESQRLVGQKTDRQTITVTERDNKLKELPDTLGELRSLRTLDISENEIQRLPQMLAHVRTLEMVLNNPVAVTSAKLSICHSGNNLAEHPSPRSPCFCESPLSSQTEEQQCLGKWQTLSLDALSMVYPPPEVCGAGTAAVQQFLCKESGLDYYPPSQYLLPVLEQDGAENSQDSPDGPTRRFSREEAEWQNRFSDYEKRKEQKMLEKLEFERRLDLGQREHAELLQQSHSHKDEILQTVKQEQTRLEQGLSERQRCLDAERQQLQEQLKQSEQSIASRIQRLLQDNQRQKKSSEILKSLENERIRMEQLMSITQEETENLRQREIAAAMQQMLTESCKSRLIQMAYESQRQSLVQQACSSMAEMDKRFQQILSWQQMDQNKAISQILQEARMLLAVDYKHAMCPVLSLLKAVSYRQQQLNPIHFRLDVELRTQDWRPLFVLLSLVFGAVLVPPVVSGALLRLQNASHLAVCSQRHVDVSDERLTSEPPLFILSVMQKAAFEALQVKKDLTHRQIRSQIRLIETELLQLTQLELKRKSLDTETLQGGCSSAPDTGFSGTQRAGPAPVEQMWSMGKGSSVQGEREMVSEQRWALSNLLQQLLKEKKQREEELHGILAELEAKSETKQENYWLIQYQRLLNQKPLSLKLQEEGMERQLVALLVELSAEHYLPLFAHHRITLDMLSRMGPGDLAKVGVSEAGLQHEILRRARDLLDVARVQPELKPPKNEVFGVSEPPTAPQELPESVRPSAPPAELDVPTSECVVCLEREAQMVFLTCGHVCCCQQCCQPLRTCPLCRQEISQRLRIYHSS

SEQ ID NO: 7 - protein, 234 aa, Homo sapiens
  misc_feature: Active portion of human Tal
LKRKSLDTESLQEMISEQRWALSSLLQQLLKEKQQREEELREILTELEAKSETRQENYWLIQYQRLLNQKPLSLKLQEEGMERQLVALLEELSAEHYLPIFAHHRLSLDLLSQMSPGDLAKVGVSEAGLQHEILRRVQELLDAARIQPELKPPMGEVVTPTAPQEPPESVRPSAPPAELEVQASECVVCLEREAQMIFLNCGHVCCCQQCCQPLRTCPLCRQDIAQRLRIYHSS

SEQ ID NO: 8 - protein, 77 aa, Homo sapiens
  misc_feature: Active portion of human Tal
VTPTAPQEPPESVRPSAPPAELEVQASECVVCLEREAQMIFLNCGHVCCCQQCCQPLRTCPLCRQDIAQRLRIYHSS

SEQ ID NO: 9 - DNA, 25 bp, Artificial sequence
  Single strand DNA oligonucleotide
ggaattcgtcatggcggtgtcggag

SEQ ID NO: 10 - DNA, 29 bp, Artificial sequence
  Single strand DNA oligonucleotide
cctcgagtcagtagaggtcactgagaccg

SEQ ID NO: 11 - DNA, 29 bp, Artificial sequence
  Single strand DNA oligonucleotide
ggaattcgggcttattcaggtcatgattg

SEQ ID NO: 12 - DNA, 25 bp, Artificial sequence
  Single strand DNA oligonucleotide
ccgggacattcccacagctccctta

SEQ ID NO: 13 - DNA, 35 bp, Artificial sequence
  Single strand DNA oligonucleotide
aaactgcagccagagcagaactgagttcttcatcc

SEQ ID NO: 14 - DNA, 27 bp, Artificial sequence
  Single strand DNA oligonucleotide
aaactgcagggcacgatccatttcctc

SEQ ID NO: 15 - DNA, 19 bp, Artificial sequence
  Single strand DNA oligonucleotide
cctgcagagctggaggtgc

SEQ ID NO: 16 - DNA, 20 bp, Artificial sequence
  Single strand DNA oligonucleotide
gacgacctcacccattggtg

SEQ ID NO: 17 - DNA, 24 bp, Artificial sequence
  Single strand DNA oligonucleotide
gtatgtattacctctataaggcac

SEQ ID NO: 18 - DNA, 23 bp, Artificial sequence
  Single strand DNA oligonucleotide
gggcttattcaggtcatgattgt

SEQ ID NO: 19 - DNA, 23 bp, Artificial sequence
  Single strand DNA oligonucleotide
cacaatcatgacctgaataagcc

SEQ ID NO: 20 - DNA, 20 bp, Artificial sequence
  Single strand DNA oligonucleotide
gaggacaccatccgagcctc

SEQ ID NO: 21 - DNA, 20 bp, Artificial sequence
  Single strand DNA oligonucleotide
gaggctcggatggtgtcctc

SEQ ID NO: 22 - DNA, 22 bp, Artificial sequence
  Single strand DNA oligonucleotide
cattcccacagctcccttatac

SEQ ID NO: 23 - DNA, 22 bp, Artificial sequence
  Single strand DNA oligonucleotide
gtataagggagctgtgggaatg

SEQ ID NO: 24 - DNA, 21 bp, Artificial sequence
  Single strand DNA oligonucleotide
ggaggtggagactacaaggac

SEQ ID NO: 25 - DNA, 24 bp, Artificial sequence
  Single strand DNA oligonucleotide
ccgggatccatggcggtgtcggag

SEQ ID NO: 26 - DNA, 37 bp, Artificial sequence
  Single strand DNA oligonucleotide
atagtttagcggccgctagtcacttgtcatcgtcgtc

SEQ ID NO: 27 - DNA, 26 bp, Artificial sequence
  Single strand DNA oligonucleotide
cccaagcttggaaggatgccgctctt

SEQ ID NO: 28 - DNA, 61 bp, Artificial sequence
  Single strand DNA oligonucleotide
ggggtacccctcatcaggcataatcgggtacatcatagggatagctgctgtggtagatgcg

SEQ ID NO: 29 - DNA, 20 bp, Artificial sequence
  Single strand DNA oligonucleotide
ctcttcttgcagcttcaagg

SEQ ID NO: 30 - DNA, 18 bp, Artificial sequence
  Single strand DNA oligonucleotide
gccaggatccagccagag

SEQ ID NO: 31 - DNA, 29 bp, Artificial sequence
  Single strand DNA oligonucleotide
cctcaactgtggcgccgtctgctgctgcc

SEQ ID NO: 32 - DNA, 29 bp, Artificial sequence
  Single strand DNA oligonucleotide
ggcagcagcagacggcgccacagttgagg

SEQ ID NO: 33 - DNA, 19 bp, Artificial sequence
  Single strand DNA oligonucleotide
cctgcagagctggaggtgc

SEQ ID NO: 34 - DNA, 20 bp, Artificial sequence
  Single strand DNA oligonucleotide
gacgacctcacccattggtg

SEQ ID NO: 35 - DNA, 19 bp, Artificial sequence
  Single strand DNA oligonucleotide
gaggagctgtcggctgagc

SEQ ID NO: 36 - DNA, 27 bp, Artificial sequence
  Single strand DNA oligonucleotide
taacttaatctggctcctgatctgccg

SEQ ID NO: 37 - protein, 19 aa, Homo sapiens
  misc_feature: Active portion of human Tal
VTPTAPQEPPESVRPSAPP

SEQ ID NO: 38 - DNA, 700 bp, Homo sapiens
  misc_feature: Active portion of human Tal
aaagaggaagtccctggacacagagtcactccaggagatgatctcggagcagcgctgggccctcagctccctgctccagcagctgctcaaagagaagcagcagcgagaggaagagctccgggaaatcctgacggagttagaagccaaaagtgaaaccaggcaggaaaattactggctgattcagtatcaacggcttttgaaccagaagcccttgtccttgaagctgcaagaagaggggatggagcgccagctggtggccctcctggaggagctgtcggctgagcactacctgcccatctttgcgcaccaccgcctctcactggacctgctgagccaaatgagcccaggggacctggccaaggtgggcgtctcagaagctggcctgcagcacgagatcctccggagagtccaggaactgctggatgcagccaggatccagccagagctgaaaccaccaatgggtgaggtcgtcacccctacggccccccaggagcctcctgagtctgtgaggccatccgctccccctgcagagctggaggtgcaggcctcagagtgtgtcgtgtgcctggaacgggaggcccagatgatcttcctcaactgtggccacgtctgctgctgccagcagtgctgccagccactgcgcacctgcccgctgtgccgccaggacatcgcccagcgcctccgcatctaccacagcagc

SEQ ID NO: 39 - DNA, 231 bp, Homo sapiens
  misc_feature: Active portion of human Tal
gtcacccctacggccccccaggagcctcctgagtctgtgaggccatccgctccccctgcagagctggaggtgcaggcctcagagtgtgtcgtgtgcctggaacgggaggcccagatgatcttcctcaactgtggccacgtctgctgctgccagcagtgctgccagccactgcgcacctgcccgctgtgccgccaggacatcgcccagcgcctccgcatctaccacagcagc

SEQ ID NO: 40 - DNA, 55 bp, Homo sapiens
  misc_feature: Active portion of human Tal
gtcacccctacggccccccaggagcctcctgagtctgtgaggccatccgctcccc

SEQ ID NO: 41 - DNA, 21 bp, Artificial sequence
  SiRNA synthetic oligonucleotide
ccuccagucuucucucguctt

SEQ ID NO: 42 - DNA, 21 bp, Artificial sequence
  SiRNA synthetic oligonucleotide
ttggaggucagaagagagcag

SEQ ID NO: 43 - DNA, 21 bp, Artificial sequence
  SiRNA synthetic oligonucleotide
guccaaagguuccggagactt

SEQ ID NO: 44 - DNA, 21 bp, Artificial sequence
  SiRNA synthetic oligonucleotide
ttcagguuuccaaggccucug

SEQ ID NO: 45 - DNA, 21 bp, Artificial sequence
  SiRNA synthetic oligonucleotide
ucaccucacuucccugcuutt

SEQ ID NO: 46 - DNA, 21 bp, Artificial sequence
  SiRNA synthetic oligonucleotide
ttaguggagugaagggacgaa

SEQ ID NO: 47 - DNA, 21 bp, Artificial sequence
  SiRNA synthetic oligonucleotide
ugcugacugagagcuguaatt

SEQ ID NO: 48 - DNA, 21 bp, Artificial sequence
  SiRNA synthetic oligonucleotide
uuacagcucucagucagcatt

SEQ ID NO: 49 - DNA, 21 bp, Artificial sequence
  SiRNA synthetic oligonucleotide
aaugucgagagucagucgutt

SEQ ID NO: 50 - DNA, 21 bp, Artificial sequence
  SiRNA synthetic oligonucleotide
acgacugacucucgacauutt

SEQ ID NO: 51 - protein, 23 aa, Artificial sequence
  PTAP-PSAP motif synthetic peptide GFP-fusion peptide
EVVTPTAPQEPPESVRPSAPPAE

SEQ ID NO: 52 - DNA, 28 bp, Artificial sequence
  Single strand DNA oligonucleotide
aagaattcagaggtcgtcacccctacgg

SEQ ID NO: 53 - DNA, 25 bp, Artificial sequence
  Single strand DNA oligonucleotide
aaggatccctctgcagggggagcgg

(19) United States
(12) Patent Application Publication
(10) Pub. No.: US 2007/0077628 A1
(43) Pub. Date: Apr. 5, 2007

(54) Polynucleotides, polypeptides and antibodies and use thereof in treating tsg101-associated diseases

(76) Inventors: Yosef Yarden, Rechovot (IL); Ido Amit, Tzur-Moshe (IL); Liat Yakir, Gan-Yavne (IL)

Correspondence Address: Martin D. Moynihan;PRTSI, Inc., P.O. BOX 16446, Arlington, VA, 22215, US

(21) Appl. No.: 10/568,707

(22) PCT Filed:
(86) PCT No.: PCT/IL04/00760

Related U.S. Application Data
(60) Provisional application No. 60/496,393, filed on Aug. 20, 2003.

(51) Int. Cl. C12Q 1/68; C12P 19/34

(52) U.S. Cl. 435/91.1; 435/6

(57) ABSTRACT

An isolated polynucleotide is provided. The isolated polynucleotide encodes a polypeptide having a sequence of at least 10 and no more than 500 amino acids, wherein said sequence is derived from the amino acid sequences of SEQ ID NO: 2, 4 or 6. Also provided are compositions and methods using same for treating Tsg101 associated diseases.

### FIELD AND BACKGROUND OF THE INVENTION

The present invention relates to polynucleotides, polypeptides and antibodies which can be used to treat TSG101-associated diseases such as AIDS.

The human immunodeficiency virus (HIV) is the agent responsible for the slow degeneration of the immune system in patients suffering from acquired immune deficiency syndrome (AIDS) [Barre-Sinoussi, F., et al., (1983) Science 220:868-870; Gallo, R., et al., (1984) Science 224:500-503]. At least two distinct types of HIV are known to date, including HIV-1 [Barre-Sinoussi, F., et al., (1983), Science 220:868-870; Gallo, R., et al., (1984), Science 224:500-503] and HIV-2 [Clavel, F., et al., (1986), Science 233:343-346; Guyader, M., et al., (1987), Nature 326:662-669]. Each of these types of viruses displays significant intra-population heterogeneity.

In humans, HIV replication occurs predominantly in CD4⁺ T lymphocyte populations, and thus leads to depletion of this cell type and eventually to immune incompetence, opportunistic infections, neurological dysfunctions, neoplastic growth, and ultimately death.

HIV is a member of the lentivirus family of retroviruses [Teich, N., et al., (1984) RNA Tumor Viruses, Weiss, R., et al., eds., CSH-Press, pp. 949-956], which are small enveloped viruses that contain a single-stranded RNA genome, and replicate via a DNA intermediate produced by a virally-encoded reverse transcriptase, an RNA-dependent DNA polymerase [Varmus, H., (1988) Science 240:1427-1439]. The HIV viral particle includes a viral core, composed in part of capsid proteins, which are associated with the viral RNA genome and enzymes required for early replicative events. A myristylated gag protein forms an outer shell around the viral core, which is, in turn, surrounded by a lipid membrane envelope derived from the infected cell membrane. The HIV envelope surface glycoproteins are synthesized as a single 160 kDa precursor protein which is cleaved by a cellular protease during viral budding into two glycoproteins, gp41 which is a transmembrane glycoprotein and gp120 which is an extracellular glycoprotein which remains non-covalently associated with gp41, possibly in a trimeric or multimeric form [Hammarskjold, M., and Rekosh, D., (1989) Biochem. Biophys. Acta 989:269-280].

Since HIV infection is pandemic, HIV-associated diseases represent a major world health problem.

Several stages of the HIV life cycle have been considered targets for therapeutic intervention [Mitsuya, H., et al., 1991, FASEB J. 5:2369-2381]. Attention has been drawn mainly to viral proteins such as, for example, the virally encoded reverse-transcriptase, as potential drug targets. A number of reverse-transcriptase-targeted drugs, including 2′,3′-dideoxynucleoside analogs such as AZT™, ddI™, ddC™, and d4T™ have shown to be effective in at least partially halting HIV replication [Mitsuya, H., et al., (1991) Science 249:1533-1544).

New treatment regimens for HIV-1 combine anti-HIV compounds, which target reverse transcriptase (RT) combined with an HIV-1 protease inhibitor. Such treatment regimens have a far greater effect on viral load (2 to 3 fold reduction) as compared to therapy using a single agent [Perelson, A. S., et al., (1996), Science 15:1582-1586].

Although such combined treatments have been somewhat effective in decreasing viral loads in AIDS patients, it is likely that long-term use thereof will lead to toxicity, especially to bone marrow cells and to the release of suppressive factors, notably the chemokines Rantes, MIP-1α and MIP-1β [Cocchi, F., et al., (1995) Science 270:1811-1815]. These effects can lead to suppression of CD8⁺ T cells, which are essential to the control of HIV, via killer cell activity [Blazevic, V., et al., (1995) AIDS Res. Hum. Retroviruses 11:1335-1342].

Another limitation inherent to long-term chemical therapy is the development of HIV mutations with partial or complete resistance [Lange, J. M., (1995) AIDS Res. Hum. Retroviruses 10:S77-82]. It is thought that such mutations may be an inevitable consequence of anti-viral therapy. The pattern of disappearance of wild-type virus and appearance of mutant virus due to treatment, combined with coincidental decline in CD4⁺ T cell numbers strongly suggests that, at least with some compounds, the appearance of viral mutants is a major factor underlying the failure of AIDS therapy.

Treatment regimens which target viral entry into the cell, which is the earliest stage of HIV infection have also been attempted. Recombinant soluble CD4, for example, has been shown to inhibit infection of CD4⁺ T cells by some HIV-1 strains [Smith, D. H., et al., (1987) Science 238:1704-17071]. Certain primary HIV-1 isolates, however, are relatively less sensitive to inhibition by recombinant CD4 [Daar, E., et al., (1990) Proc. Natl. Acad. Sci. USA 87:6574-6579]. In addition, recombinant soluble CD4 clinical trials have produced inconclusive results [Schooley, R., et al., (1990) Ann. Int. Med. 112:247-253; Kahn, J. O., et al. (1990) Ann. Int. Med. 112:254-261; Yarchoan, R., et al. (1989) Proc. Vth Int. Conf. on AIDS, p. 564, MCP 137].

The late stages of HIV replication, which involve crucial virus-specific processing of certain viral encoded proteins, have also been suggested as possible anti-HIV drug targets. Late stage processing is dependent on the activity of a viral protease, therefore drugs designed for inhibiting this protease are currently in late developmental stages [Erickson, J. (1990) Science 249:527-533], although the predicted therapeutic potential of such drugs is questionable.

In view of the limited number of therapeutic approaches available and the large number of infected individuals world wide (forty million people according to UNAIDS), there is a widely recognized need for novel approaches for treating AIDS and halting the spread of this deadly disease.

Recently attention of the HIV/AIDS research community has been drawn to understanding the cross talk between viral proteins and host factors. Of particular, interest are host proteins which act late in the viral assembly and release pathway. This step of viral infection is driven by the viral Gag precursor, Pr55^{Gag} [Freed (2002) J. Virol. 76:4679-87], which attaches to the inner leaflet of the plasma membrane and multimerizes to trigger the budding of virions. Following release from the cell, Pr55^{Gag}, undergoes cleavage to generate several proteins including the matrix, capsid, nucleocapsid and p6 proteins.

Studies of retroviral particles indicated that the Gag protein harbors a late, or ‘L’ domain, whose disruption results in a phenotype characterized by virion assembly that is normal but devoid of the late budding event [Willis and Craven (1991) AIDS 5, 639-654]. Because L-domains are autonomous and independent of their position within Gag, it has been proposed that they recruit host factors necessary for budding. Indeed, each of the retroviral L-domains characterized to date contains one of three sequence motif that bind cellular proteins: a P(T/S)AP tetrapeptide binds Tsg101 [VerPlank et al., (2001) Proc Natl Acad Sci U S A 98, 7724-9], PPXY binds the ubiquitin ligase Nedd4 [Kikonyogo and et al. (2001) Proc Natl Acad USA 98, 11199-11204], and a YXXL motif binds the clathrin adaptor AP2 [Puffer et al. (1997) J Virol 71, 6541-6546].

The PTAP motif of HIV-1, which is conserved in all HIV and SIV strains binds Tsg101, a component of the vesicular sorting machinery [Babst et al. (2000) Traffic 1, 248-58].

Tsg101 participates in endosome maturation by controlling budding of vesicles into the endosome lumen to create the multivesicular body (MVB). A topologically similar budding (i.e., ‘away from the cytoplasm’) occurs when viruses bud at the plasma membrane. The mammalian tumor susceptibility 101 (tsg101) gene was initially discovered in a screen for tumor suppressor genes [Li and Cohen (1996) Cell 85:319-329], whereas the yeast ortholog was identified by virtue of its ability to induce a class E compartment representing a defect in endosome maturation and MVB formation [Katzmann et al. (2002) Nat Rev Mol Cell Biol 3, 893-905].

Depletion of Tsg101 from HIV-producing cells results in a budding defect, whereas a short peptide motif can restore budding competence to a late domain-defective HIV, consistent with the essential role of Tsg101 in HIV egress [Demirov et al. (2002) Proc Natl Acad Sci U S A 99, 955-60; Gamis et al. (2001) Cell 107, 55-65; Martin-Serrano et al. (2001) Nat Med 7, 1313-9].

While reducing the present invention to practice the present inventors have uncovered a Tsg101 associated ligase (Ta1) which attaches ubiquitin (Ub) molecules to Tsg101 to thereby regulate release of HIV particles from infected cells. These findings indicate an essential and important role for Ta1 in controlling HIV budding and, as such, serve as a basis for an agent suitable for controlling viral spread and disease progression.

### SUMMARY OF THE INVENTION

According to one aspect of the present invention there is provided an isolated polynucleotide encoding a polypeptide having a sequence of at least 10 and no more than 500 amino acids, wherein the sequence is derived from the amino acid sequences of SEQ ID NO:2, 4 or 6.

According to another aspect of the present invention there is provided a nucleic acid construct comprising the isolated polynucleotide.

According to further features in preferred embodiments of the invention described below, further comprising a promoter for regulating transcription of the isolated polynucleotide in sense or antisense orientation.

According to still further features in the described preferred embodiments the nucleic acid construct further comprises a positive and a negative selection markers for selecting for homologous recombination events.

According to yet another aspect of the present invention there is provided a host cell comprising the nucleic acid construct.

According to still another aspect of the present invention there is provided an isolated polynucleotide as set forth in SEQ ID NO:38, 39 or 40.

According to an additional aspect of the present invention there is provided an isolated polypeptide comprising an amino acid sequence of at least 10 and no more than 500 amino acids, wherein the amino acid sequence is derived from SEQ ID NO: 2, 4 or 6.

According to still further features in the described preferred embodiments the amino acid sequence is as set forth in SEQ ID NO:7, 8, 37 or 51.

According to yet an additional aspect of the present invention there is provided an antibody or an antibody fragment being capable of specifically binding a polypeptide at least 90% homologous to SEQ ID NO: 2, as determined using the BestFit software of the Wisconsin sequence analysis package, utilizing the Smith and Waterman

According to still an additional aspect of the present invention there is provided a display library comprising a plurality of display vehicles each displaying at least 6 consecutive amino acids derived from a polypeptide at least 90% homologous to SEQ ID NOs: 2 as determined using the BestFit software of the Wisconsin sequence analysis package, utilizing the Smith and Waterman algorithm, where gap creation penalty equals 8 and gap extension penalty equals 2.

According to a further aspect of the present invention there is provided an oligonucleotide specifically hybridizable with a nucleic acid sequence as set forth in SEQ ID NO: 1.

According to still further features in the described preferred embodiments the oligonucleotide is a single or double stranded.

According to still further features in the described preferred embodiments the oligonucleotide is at least 10 bases long.

According to still further features in the described preferred embodiments the oligonucleotide is hybridizable in either sense or antisense orientation.

According to yet a further aspect of the present invention there is provided a pharmaceutical composition comprising a therapeutically effective amount of at least an active portion of a polypeptide being at least 90% homologous to SEQ ID NO: 2, as determined using the BestFit software of the Wisconsin sequence analysis package, utilizing the Smith and Waterman algorithm, where the gap creation equals 8 and gap extension penalty equals 2 or an active portion thereof and a pharmaceutically acceptable carrier or diluent.

According to still a further aspect of the present invention there is provided a method of treating HIV infection in a subject, the method comprising providing to a subject in need thereof a therapeutically effective amount of at least an active portion of a polypeptide being at least 90% homologous to SEQ ID NO: 2, as determined using the BestFit software of the Wisconsin sequence analysis package, utilizing the Smith and Waterman algorithm, where the gap creation equals 8 and gap extension penalty equals 2 or an active portion thereof, to thereby treat the HIV infection in the subject.

According to still further features in the described preferred embodiments providing is effected by:
(i) administering the polypeptide to the subject; and/or
(ii) administering an expressible polynucleotide encoding the polypeptide to the subject.

According to still further features in the described preferred embodiments the method further comprises providing to the subject a therapeutically effective amount of Tsg101.

According to still a further aspect of the present invention there is provided a nucleic acid construct system comprising: (a) a first nucleic acid construct including a first polynucleotide encoding at least an active portion of a polypeptide being at least 90% homologous to SEQ ID NO: 2, as determined using the BestFit software of the Wisconsin sequence analysis package, utilizing the Smith and Waterman algorithm, where the gap creation equals 8 and gap extension penalty equals 2; and (b) a second nucleic acid construct including a second polynucleotide encoding Tsg101 or an active portion thereof.

According to still further features in the described preferred embodiments the first polynucleotide is as set forth in SEQ ED NO: 1, 3 or 5.

According to still a further aspect of the present invention there is provided a nucleic acid construct comprising a first polynucleotide encoding at least an active portion of a polypeptide being at least 90% homologous to SEQ ID NO: 2, as determined using the BestFit software of the Wisconsin sequence analysis package, utilizing the Smith and Waterman algorithm, where the gap creation equals 8 and gap extension penalty equals 2 and a second polynucleotide encoding Tsg101.

According to still further features in the described preferred embodiments the polypeptide is as set forth in SEQ ID NO:2, 4 or 6.

According to still further features in the described preferred embodiments the nucleic acid construct system of 36, wherein the first polynucleotide is at least 85% identical to SEQ ID NO: 1, as determined using the BestFit software of the Wisconsin sequence analysis package, utilizing the Smith and Waterman algorithm, where gap weight equals 50, length weight equals 3, average match equals 10 and average mismatch equals −9.

According to still further features in the described preferred embodiments the first polynucleotide is as set forth in SEQ ID NO:1, 3 or 5.

According to still further features in the described preferred embodiments the active portion of the polypeptide is as set forth in amino acid coordinates 490-723 of SEQ ID NO:2.

According to still further features in the described preferred embodiments the active portion of the polypeptide is as set forth in amino acid coordinates 647-723 of SEQ ID NO:2.

According to still further features in the described preferred embodiments the active portion of the polypeptide is as set forth in amino acid coordinates 647-665 of SEQ ID NO:2.

According to still further features in the described preferred embodiments the active portion of the polypeptide is as set forth in amino acid coordinates 647-667 of SEQ ID NO:2.

According to still further features in the described preferred embodiments the active portion of the polypeptide is encoded by nucleotide coordinates 1556-2255 of SEQ ID NO:1.

According to still further features in the described preferred embodiments the active portion of the polypeptide is encoded by nucleotide coordinates 2025-2255 of SEQ ID NO:1.

According to still further features in the described preferred embodiments the active portion of the polypeptide is encoded by nucleotide coordinates 2025-2079 of SEQ ID NO:1.

According to still further features in the described preferred embodiments the active portion of the polypeptide is encoded by nucleotide coordinates 2019-2088 of SEQ ID NO:1.

According to still further features in the described preferred embodiments the nucleic acid construct further comprises a promoter for regulating transcription of the first and second polynucleotides in sense or antisense orientation.

According to still further features in the described preferred embodiments the promoter is active in a mammalian cell.

According to still a further aspect of the present invention there is provided a method of treating HIV infection and/or a hyperproliferative disease associated with disregulated activity of Tsg101 in a subject, the method comprises downregulating in a subject in need thereof a polypeptide being at least 90% homologous to SEQ ID NO: 2, as determined using the BestFit software of the Wisconsin sequence analysis package, utilizing the Smith and Waterman algorithm, where the gap creation equals 8 and gap extension penalty equals 2, to thereby treat the HIV infection in the subject.

According to still further features in the described preferred embodiments downregulating the polynucleotide is effected using a ribozyme being specifically hybridizable with the polynucleotide.

According to still further features in the described preferred embodiments downregulating the polynucleotide is effected using an antisense being specifically hybridizable with the polynucleotide.

According to still further features in the described preferred embodiments downregulating the polynucleotide is effected using a small interfering RNA duplex being specifically hybridizable with the polynucleotide.

According to still further features in the described preferred embodiments the small interfering RNA duplex is set forth in SEQ ID NOs:45 and 46.

According to still further features in the described preferred embodiments the small interfering RNA duplex is set forth in SEQ ID NOs:47 and 48.

According to still further features in the described preferred embodiments downregulating the polypeptide is effected using an antibody.

The present invention successfully addresses the shortcomings of the presently known configurations by providing novel polynucleotides, polypeptides and antibodies which can be used in methods of treating TSG101-associated diseases such as AIDS.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1a is a schematic diagram depicting Tsg101, Ta1 and Gag regions expressed in yeast as C-terminal fusion proteins.

FIG. 1b is a histogram depicting binding of Tsg101 to Ta1 and Gag proteins, as determined using reporter gene activity assays. Yeast were transformed with different plasmids encoding the indicated proteins. Reporter gene activation was measured by growth on selective medium (-trp-leu-his, not shown) and by liquid β-galactosidase assay of the yeast lysates using ONPG [O-Nitrophenyl β-D-Galactopyranoside] as a substrate. Data is represented as the mean β-galactosidase activity of three separate assays using independent transformants.

FIG. 1c is a histogram depicting binding of Tsg101 to Ta1 proteins, effected as in FIG. 1b.

FIG. 1d—is a photomicrograph depicting cell growth of yeast transformed with wild type or deletion mutants of Ta1 and Tsg101 following serial dilutions and plating on a selective (Trp-Leu-His minus) medium.

FIG. 2a is the amino acid sequence of human Ta1. The two P(T/S)AP motifs are highlighted.

FIG. 2b is a schematic illustration of human Ta1 depicting the approximate boundaries of the leucine-rich repeats (LRR), ezrin-radixin-moesin (ERM) domain, coiled-coil (CC) region, a sterile α motif (SAM) and a RING-finger motif.

FIG. 2c is a multiple sequence alignment of human, rat, mouse and Ciona intestinalis Ta1. Conserved sequences are highlighted.

FIG. 2d is a western blot analysis showing expression of Ta1 protein in cell lines and tissues. Ta1 was immunoprecipitated (IP) from extracts of HEK-293T cells transfected with either the respective plasmid, or an empty vector. Alternatively, extracts of mouse brain were subjected to IP and immunoblotting (IB) with antibodies directed to the indicated amino acids (AA) of hTa1.

FIG. 2e is a northern blot analysis showing expression of Ta1 in various tissues. Poly-adenylated RNA was extracted from the indicated mouse tissues, resolved by electrophoresis, and the Northern blot was hybridized to a human Ta1 cDNA probe. The location of a 4 kb marker is shown. Equal lane loading (approximately 2 micrograms of RNA) was verified by RNA staining (not shown).

FIG. 3a is pull down assay depicting the interaction between Ta1 and Tsg101 in mammalian cells. Whole extracts derived from HEK-293T cells co-expressing mGST-Tsg101 and hTa1 (HA-tagged) were subjected to a pull down (PD) assay using glutathione-agarose beads and immunoblotting (IB).

FIG. 3b is a co-immunoprecipitation analysis depicting the interaction between Ta1 and Tsg101 in mammalian cells. Whole extracts derived from HEK-293T transiently expressing HA-hTa1 were subjected to immunoprecipitation with an antibody directed to the endogenous Tsg101 protein. Cell lysates and immunoprecipitates were analyzed by western blotting using anti HA antibodies or anti Tsg101 antibodies.

FIG. 3c is a co-immunoprecipitation analysis depicting the interaction between Ta1 and Tsg101 in mammalian cells. HEK-293T cells co-expressing hTa1 (HA-tagged) and a Flag-tagged Tsg101, either wild type or the indicated deletion mutants, were analyzed by immunoprecipitation (IP) and IB using anti Flag antibodies or anti HA antibodies.

FIGS. 4a-b are photomicrographs which illustrate that human Ta1 increases ubiquitylation of Tsg101 in a RING finger-dependent manner. HEK-293T cells were co-transfected with plasmids encoding a Flag-tagged TSG-101 (either wild type or a mutant lacking the steadiness box), HA-tagged hTa1 [either wild type (+) or a RING mutant (H695A)], and Myc-tagged ubiquitin. Forty-eight hours following transfection, cells were divided into two unequal portions: the first portion (75% of cell population) was extracted in. Triton X-100 (1%; weight/volume, FIG. 4a), and the remainder (25% of cell population) was extracted in SDS (1%; weight/volume, FIG. 4b). Following removal of insoluble material, the concentration of SDS was reduced to 0.1% by adding Triton X-100-containing lysis buffer. Immunoblotting of immunoprecipitates or whole extracts was performed with the indicated antibodies.

FIG. 4c shows ubiquitination of Tsg101 proteins in the soluble cell fraction by the indicated Ta1 mutants. HEK-293T cells transiently expressing the indicated proteins, along with Myc-ubiquitin, were lysed in 1% Triton X-100 and subjected to immunoprecipitation with anti-Flag antibody and immunoblotting.

FIG. 4d shows ubiquitination of Tsg101 proteins in the insoluble cell fraction by the indicated Ta1 mutants. The insoluble material from the experiment shown in FIG. 4c was solubilized with 1% SDS containing lysis buffer. Prior to immunoprecipitation with anti-Flag antibody, the SDS concentration was diluted to 0.1% with Triton X-100 lysis buffer. Immunoblots were performed using anti Flag antibodies, anti Myc-Ub antibodies or anti HA antibodies.

FIGS. 5a-e are photomicrographs depicting Ta1 dependent ubiquitylation of Tsg101. FIGS. 5a-b are photomicrographs depicting Ta1 dependent ubiquitylation of Tsg101 as determined by western blot analysis in transiently transfected HEK-293T cells. HEK-293T cells transiently expressing the wild type Tsg101 and either wild-type (WT) or truncated forms of Ta1, along with Myc- (FIG. 5a) or Flag- (FIG. 5b) tagged ubiquitin, were extracted in Triton X-100 and subjected to western blot analysis. FIGS. 5c-d—are autoradiograms depicting Ta1 dependent mono-ubiquitylation of Tsg101 as determined by in-vitro ubiquitylation assay. FIG. 5c—WT or mutant forms of HA-Ta1 were expressed in HEK-293T cells, immobilized on anti-HA-decorated agarose beads, and incubated with recombinant E1, E2 (Ubc-H5B) and ¹²⁵I-labelled ubiquitin. Ubiquitylated products were resolved by gel electrophoresis and detected by autoradiography. FIG. 5d—Flag-Tsg101 was immobilized by using anti-Flag antibodies, and incubated with an ubiquitylation assay mixture supplemented with whole cell extracts. Extracts were derived from HEK-293T cells transfected with a control plasmid (Con) or vectors encoding the indicated forms of hTa1. After extensive washing, the Tsg101-hTa1 complex was subjected to ubiquitylation in vitro. A control reaction was performed in the absence of cell extract (lane labeled —). FIG. 5e is a photomicrograph depiction of Ta1-mediated ubiquitylation of GST-Tsg101 Extracts of HEK-293T cells expressing mGST-Tsg101, Ta1-HA (either WT or C675A), and Flag- or Myc- tagged ubiquitin (either WT or 4KR) were subjected to pull down (PD) with glutathione beads. A portion (10%) of the beads was then analyzed and the rest eluted at 95° C., and subjected to IP with an anti-Flag antibody. Open arrows indicate the unmodified mGST-Tsg101, and filled arrows indicate the mono-ubiquitylated species. Note that the second isolation step yielded no Myc-reactive mono- and di-ubiquitylated Tsg101.

FIGS. 6a-c are photomicrographs depicting a partial co-localization of Ta1 with Tsg101 and Gag at a sub-membranal domain. FIG. 6a—HeLa cells that over-express Flag-Tsg101, along with HA-hTa1 and EGFR, were pre-incubated for 45 minutes at 4° C. with EGF conjugated to AlexaFluor⁴⁸⁸. Cells were fixed, permeabilized, and stained with primary and fluorescently-labeled secondary antibodies, prior to confocal microscopy. The merge panel shows all three probes. FIG. 6b—HeLa cells expressing HA-hTa1 (either WT or ΔCC) and Flag-Tsg101 (either WT or ΔSB) were visualized as in FIG. 6a. FIG. 6c—Cultures of HeLa-SS6 cells grown on poly-D-lysine-coated glass slides were transfected with plasmids encoding either WT or C675A HA-hTa1. Cells were treated 24 hours after transfection with either a control vector (left column) or a Gag-GFP-encoding plasmid. Fixation, staining and confocal visualization were performed six hours later. Bars represent 20 microns.

FIGS. 7a-h are photomicrographs showing a synergistic inhibitory activity of Ta1 and TSG101 in the release of HIV-1 virus like particles (VLPs) and infectious virions. FIG. 7a—HEK-293T cells were co-transfected with vectors expressing HIV-1 Gag -GFP, and wildtype or mutant Ta1 and Tsg101, as indicated. Supernatants were harvested 24 hrs or 36 hrs post-transfection, and the presence of Gag in pelleted virus like particles from the supernatant or in cytoplasmic extracts was analyzed by immunobloting with anti-GFP antibody. Note the appearance of a RING mutant of hTa1 in virus-like particles 36 hours following transfection. FIGS. 7b-c—HEK-293T cells were co-transfected with the pNLenv-1 vector encoding HIV-1 Gag, along with the indicated plasmids. A Myc-Ub plasmid was used only in FIG. 7c. Forty-eight hours post-transfection, VLPs were harvested, cells were extracted and analyses performed either directly or after IP with anti-p24G antibodies. FIG. 7d—HEK-293T cells were co-transfected with a mixture of plasmids that generates an infectious HIV-1-based vector, and the indicated treatment constructs. Virus-containing supernatants were harvested two days later, and used to infect naive HEK-293T cells. Shown are normalized infectivity results of duplicate determinations (average±S.D.). Infectivity is reported relative to the control, where no Tsg101 and hTa1 proteins were expressed ectopically. The experiment was repeated twice. FIG. 7e—HeLa-SS6 cells expressing either WT or C675A-hTa1 were treated with either a control vector (left column), or a Gag-GFP-encoding plasmid. Fixation, staining and confocal visualization were performed six hours later. FIG. 7f—HeLa-SS6 cells were transfected with the indicated siRNA oligonucleotides, and twenty-four hours later a second transfection was performed with vectors encoding HIV-1 Gag [pNLenv-1; Schubert et al. (1995) J. Virol. 69(12):7695-711] and HA-hTa1 (or a control plasmid). Cells were extracted 24 hours later, and co-immunoprecipitation of Ta1 and Gag was tested by using the respective antibodies. FIG. 7g—HEK-293T cells were co-transfected with the pNLenv-1 vector encoding HIV-1 Gag, along with WT-hTa1 or the indicated mutants. The presence of Ta1 in VLPs was tested 48 hrs later. FIG. 7h—HeLa-SS6 cells were transfected with a Ta1-specific siRNA, which starts at nucleotide 1252, and a control inverted sequence (50 nM each). Forty-eight hours later, cells were co-transfected with the indicated oligonucleotides (25 nM) along with pNLenv-1 (1 μg). The presence of Gag in VLPs or in cytoplasmic extracts was analyzed 24 hours later.

FIGS. 8a-g show the effect of Ta1 and catalytically-inactive mutants thereof on endocytic degradation of EGF-receptors and signaling therefrom. FIG. 8a—HeLa cells expressing HA-hTa1 (either WT or H695A) were pre-incubated at 4° C. with EGF conjugated to AlexaFluor 488. Thereafter, cells were incubated at 37° C. for the indicated time intervals, fixed, permeabilized, and stained with anti-HA antibodies, followed by fluorescent secondary antibodies. FIG. 8b—Chinese hamster ovary cells transfected with plasmids encoding EGFR, Flag-Tsg101 and HA-hTa1, or the indicated mutants, were surface biotinylated 48 hours after transfection, and analyzed as indicated. FIG. 8c—HEK-293 cells stably expressing the ecdysone receptor, were transfected with plasmids that express HA-hTa1 (WT or C675A) from an ecdysone inducible promoter. The indicated stable clones were incubated at 37° C. without or with Muristerone A (2 μM) for various time intervals and cell extracts analyzed directly by IB. Numbers below lanes indicate quantification of signals normalized to the respective tubulin signal. FIG. 8d—HeLa-SS6 cells were transfected with siRNA oligonucleotides (50 nM each). Forty-eight hours post-transfection, cells were starved for six hours in the absence of serum, and then stimulated with EGF (20 ng/ml) for one hour. Whole-cell extracts were immunoblotted with the indicated antibodies. FIG. 8e—Chinese hamster ovary (CHO) cells transiently transfected with an EGFR plasmid, along with either a C675A-Ta1 (solid line) or an empty vector (Control; dashed line), were pre-incubated in cysteine- and methionine-free medium prior to a 20 minute-long pulse of [³⁵S]-labeled amino acids. Thereafter, cells were chased at 37° C. in fresh medium for the indicated time intervals. An autoradiogram of the immunoprecipitated EGFR is shown, along with the respective quantification of the precursor (p140) and mature (p170) forms of EGFR. FIG. 8f—HEK-293 cells expressing HA-hTa1 (WT or C675A) from a Muristerone-inducible promoter were incubated without or with Muristerone A (2 μM) for forty-eight hours. Cell extracts were tested for co-immunoprecipitation of hTa1 and EGFR. FIG. 8g—HEK-293T cells were co-transfected with a GFP-ERK2 plasmid, and either a vector encoding for HA-hTa1, or a control empty plasmid. Thirty-six hours post-transfection, cells were starved for eight hours in the absence of serum, and then stimulated with EGF (100 ng/ml) for the indicated time intervals. Whole-cell extracts were immunoblotted with antibodies to the doubly phosphorylated ERK (pERK) or a general ERK antibody (gERK). Shown are the resulting immunoblots (inset) and quantification of the active ERK signal.

FIGS. 9a-b depict inhibition of HIV-1 budding by a PTAP containing Ta1 peptide (SEQ ID NO: 51). FIG. 9a—shows a GFP-fusion to the PTAP containing Ta1 peptide. FIG. 9b HEK-293T cells were co-transfected with 1 μg of pNLenv-1 vector encoding HIV-1 Gag and 0.5 μg of a GFP-PTAP containing Ta1 peptide (SEQ ID NO: 51) or 0.5 μg of a empty GFP vector which was used as a control. Supernatants were harvested 36 hrs post-transfection, and the presence of Gag in pelleted virus like particles from the supernatant or in cytoplasmic extracts was analyzed by immunobloting with anti-p24^{Gag} antibodies.

FIG. 10 is a scheme depicting interactions between Ta1 and TSG101. The domain structures of Ta1 and Tsg101 are depicted, along with their intermolecular binding specificities. Note that the UEV domain of Tsg101 binds the double PTAP motif of Ta1, and a distinct site binds a monomeric ubiquitin (not presented). Secondary interactions between Ta1 and Tsg101 involve a region encompassing the coiled coil (CC) domain of Ta1 and the steadiness box (SB) of Tsg101. Potentially, both binding sites of the UEV domain may be blocked intramolecularly through binding to the C-terminally located PTAP motif and to a monomeric ubiquitin conjugated by Ta1.

FIG. 11 is a scheme depicting functional interactions between Ta1 and TSG101. The model illustrates the role of the Ta1-Tsg101 complex in budding of vesicles into the lumen of the multi-vesicular body (MVB) and in virus budding. Accordingly, Tsg101 sorts cargo proteins like the epidermal growth factor receptor (EGFR) and HIV Gag into budding structures. Ta1-mediated ubiquitylation of Tsg101 inactivates this sorting function, and concomitantly translocates Tsg101 from relatively insoluble membrane subdomains. Presumably, the coordinated action of Ta1 and a deubiquitylation enzyme (DUB) enables recycling of Tsg101 and re-loading of cargo.

### DESCRIPTION OF THE PREFERRED EMBODIMENTS

The present invention is of polynucleotides, polypeptides and antibodies, which can be used in treatment of TSG101-associated diseases such as AIDS.

The principles and operation of the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

The human immunodeficiency virus (HIV) is the primary cause of the degenerative immune system disease termed acquired immune deficiency syndrome (AIDS) [Barre-Sinoussi, F., et al., (1983) Science 220:868-870; Gallo, R., et al., (1984) Science 224:500-503]. HIV infection leads to depletion of T cells, which serve as hosts for viral replication and as such, this disease eventually leads to immune incompetence, opportunistic infections, neurological dysfunctions, neoplastic growth, and ultimately death.

Although considerable effort is being put into identifying or designing therapeutics effective in inhibiting HIV replication, current therapeutic approaches fail to eradicate the disease in infected individuals.

Recent studies have focused on the role of various host proteins in viral assembly and budding in infected cells. Such studies have shown that Tsg101, a protein component of the vesicular sorting mechanism, participates in the budding stage of HIV.

While reducing the present invention to practice the present inventors have uncovered a Tsg101 associated ligase (Ta1, SEQ ID NOs: BC009239), which attaches ubiquitin (Ub) molecules to Tsg101 to thereby inhibit release of HIV particles from infected cells.

As is illustrated in the Examples section which follows, Ta1 is a RING-finger containing protein with a unique domain structure including, an N-terminal leucine-rich repeat (LRR) domain followed by an ERM domain, coiled coil (cc) region, a SAM domain and a C-terminal C3HC4-type RING finger domain, which is present in many E3 ubiquitin ligases [Joazeiro and Weissman (2000) Cell 102:549-52]. Notably, Ta1 contains adjacent PTAP and PSAP motifs in the C-terminus thereof, which mediate binding to the UEV domain of Tsg101. Secondary interactions between Ta1 and Tsg101 involve a region encompassing the coiled coil (cc) domain of Ta1 and the steadyness box (SB) of Tsg101 (see FIG. 10). These motifs are conserved among Ta1 orthologs. Sequence alignment analysis illustrates that Ta1 proteins are highly homologous, representing a distinct protein family. Preliminary analysis suggests that the gene encoding Ta1 is represented as a single copy gene in all vertebrate genomes. Furthermore, Ta1 is ubiquitously expressed, though elevated levels of the protein were detected in a brain tissue.

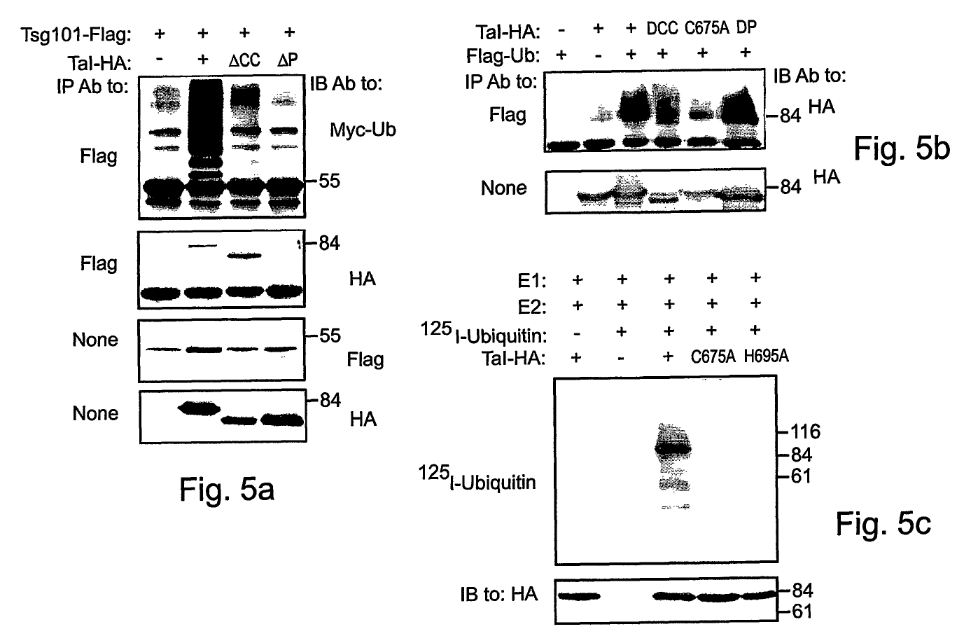

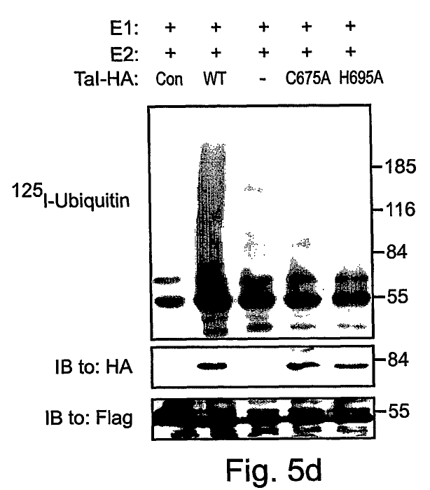

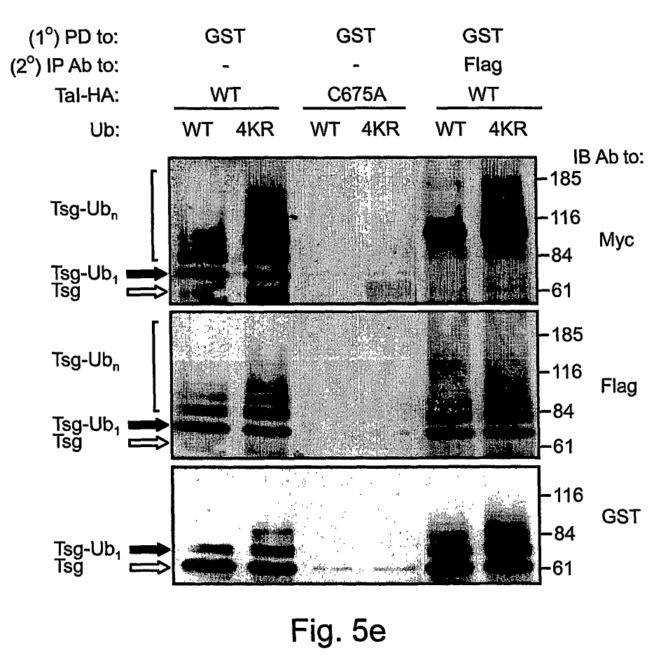

Ta1 ubiquitinates Tsg101 in a RING-finger dependent manner, as mutations at conserved residues within the RING finger domain abolish ubiquitination of Tsg101. Interestingly, ubiquitination of Tsg101 is dependent on (i) an integral steadiness box (SB) of Tsg101 and on (ii) conserved PTAP/PSAP sequence motifs of Ta1 as deletion of these sequences abolishes the ubiquitination of Tsg101 (FIG. 5a). Morphological and biochemical analyses indicated that ubiquitination of Tsg101 by Ta1 shifts Tsg101 to Triton X-100 soluble fraction.

As is shown in Example 6 of the Examples section, expression of Ta1 in cells also expressing the HIV Gag polypeptide inhibits secretion of this protein from infected cells, clearly showing that Ta1 can be used to regulate viral budding and infectivity. Notably, co-expression of Ta1 and Tsg101 elicits a synergistic inhibitory effect on Gag release.

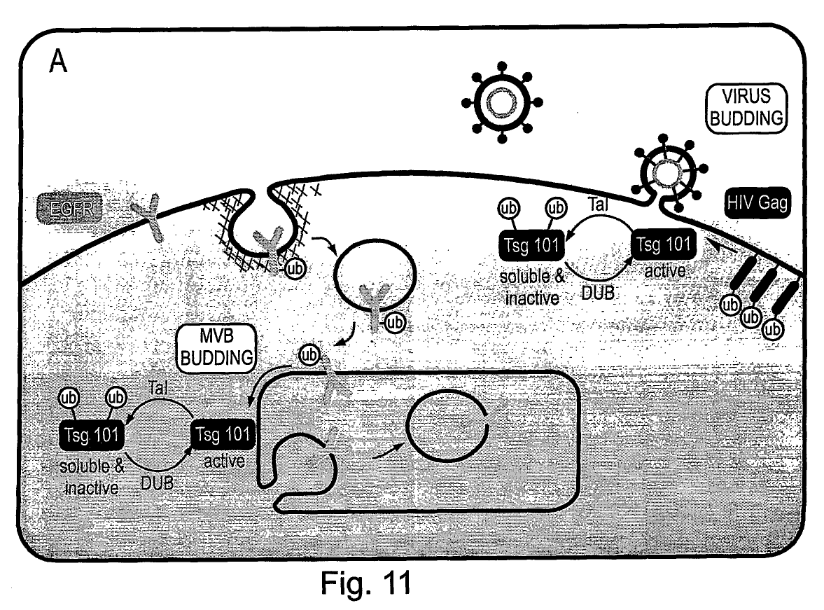

All these findings indicate an essential role for Ta1 and fragments thereof in controlling HIV budding and as such these sequences serve as a basis for use in therapeutic formulation for preventing viral spread and disease progression (see FIG. 11).

Thus, according to one aspect of the present invention there is provided an isolated polynucleotide encoding a polypeptide having a sequence at least 10 and no more than 500 amino acids. This sequence is derived from (i.e., obtained from) an amino acid sequence of SEQ ID NO: 2 (human Ta1, GenBank Accession No:BC009239), SEQ ID NO:4 (mouse Ta1, GenBank Accession No: XM149118.3) or SEQ ID NO:6 (rat Ta1, GenBank Accession No:XM231157.1)

The amino acid sequences of the present invention refer to peptides which display one or more functions of Ta1 (i.e., active portions thereof), including but not limited to binding of Tsg101, ubiquitination of Tsg101, translocation of Tsg101 to a soluble cellular compartment, inhibition of HIV infection, inhibition of HIV budding and EGFR degradation (see Examples 6-7 of the Examples section which follows and FIG. 11).

As mentioned hereinabove “an isolated polynucleotide” refers to a single or double stranded nucleic acid sequences which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is at least partially complementary and at least partially genomic. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements.

According to one preferred embodiment of this aspect of the present invention the amino acid sequence of the present invention includes amino acid coordinates 490-723 of SEQ ID NO: 2, which is encoded by nucleotide coordinates 1556-2255 of SEQ ID NO: 2 (SEQ ID NOs. 7 and 38). Such an amino acid sequence is capable of regulating Tsg101 activity through the addition of ubiquitin moieties to the Tsg101 molecule, thereby modulating Tsg101 localization and function, to thereby down-regulate HIV infectivity.

According to yet another preferred embodiment of this aspect of the present invention the amino acid sequence of the present invention includes amino acid coordinates 647-723 of SEQ ID NO: 2, which is encoded by nucleotide coordinates 2025-2255 of SEQ ID NO: 1 (SEQ ID NOs.8 and 39).

According to still another preferred embodiment of this aspect of the present invention the amino acid sequence of the present invention includes amino acid coordinates 647-665 of SEQ ID NO: 2, which is encoded by nucleotide coordinates 2025-2079 of SEQ ID NO: 1 (SEQ ID NOs.37 and 40).

According to an additional preferred embodiment of this aspect of the present invention the amino acid sequence of the present invention includes amino acid coordinates 645-667 of SEQ ID NO: 2, which is encoded by nucleotide coordinates 2019-2088 of SEQ ID NO: 1 (SEQ ID NO. 51). GFP peptide fusion of this sequence was shown to inhibit HIV-1 budding as described in Example 8 of the Examples section.

The isolated polynucleotides of the present invention can be ligated into a nucleic acid construct designed for expression of coding sequences in prokaryotic and/or eukaryotic cells (e.g., mammalian cells).

To enable cellular expression of the polynucleotides of the present invention, the nucleic acid construct of the present invention includes at least one cis acting regulatory element. As used herein, the phrase “cis acting regulatory element” refers to a polynucleotide sequence, preferably a promoter, which binds a trans acting regulator and regulates the transcription of a coding sequence located downstream thereto.

Any suitable promoter sequence can be used by the nucleic acid construct of the present invention.

Preferably, the promoter utilized by the nucleic acid construct of the present invention is active in the specific cell population transformed. Examples of cell type-specific and/or tissue-specific promoters include promoters such as albumin that is liver specific [Pinkert et al., (1987) Genes Dev. 1:268-277], lymphoid specific promoters [Calame et al., (1988) Adv. Immunol. 43:235-275]; in particular promoters of T-cell receptors [Winoto et al., (1989) EMBO J. 8:729-733] and immunoglobulins; [Banerji et al. (1983) Cell 33729-740], neuron-specific promoters such as the neurofilament promoter [Byrne et al. (1989) Proc. Natl. Acad. Sci. USA 86:5473-5477], pancreas-specific promoters [Edlunch et al. (1985) Science 230:912-916] or mammary gland-specific promoters such as the milk whey promoter (U.S. Pat. No. 4,873,316 and European Application Publication No. 264,166). The nucleic acid construct of the present invention can further include an enhancer, which can be adjacent or distant to the promoter sequence and can function in up regulating the transcription therefrom.

The nucleic acid construct of the present invention preferably further includes an appropriate selectable marker and/or an origin of replication. The nucleic acid construct utilized by the present invention can be a shuttle vector, which can propagate both in E. coli (wherein the construct comprises an appropriate selectable marker and origin of replication) and mammalian cells. The construct according to the present invention can be, for example, a plasmid, a bacmid, a phagemid, a cosmid, a phage, a virus or an artificial chromosome.

Examples of suitable constructs include, but are not limited to pcDNA3, pcDNA3.1 (+/−), pGL3, PzeoSV2 (+/−), pDisplay, pEF/myc/cyto, pCMV/myc/cyto each of which is commercially available from Invitrogen Co. (www.invitrogen.com). Examples of retroviral vector and packaging systems are those sold by Clontech, San Diego, Calif., including Retro-X vectors pLNCX and pLXSN, which permit cloning into multiple cloning sites and the transgene is transcribed from CMV promoter. Vectors derived from Mo-MuLV are also included such as pBabe, where the transgene is transcribed from the 5′LTR promoter.

Since the polynucleotide sequences of the present invention encode previously functionally undefined polypeptides, the present invention also encompasses an isolated polypeptide or portions thereof, which are encoded by the isolated polynucleotide and respective nucleic acid fragments thereof which are described hereinabove.

Identification of peptide regions of Ta1 which are capable of binding Tsg101 or other biomolecular targets participating in HIV budding can be effected using various approaches, including, for example, display techniques.

Thus, according to still another aspect of the present invention there is provided a display library comprising a plurality of display vehicles (such as phages, viruses or bacteria) each displaying at least 6, at least 7, at least 8, at least 9, at least 10, 10-15, 12-17, or 15-20 consecutive amino acids derived from the polypeptide sequence of Ta1.

Methods of constructing such display libraries are well known in the art. Such methods are described in, for example, Young AC, et al., “The three-dimensional structures of a polysaccharide binding antibody to Cryptococcus neoformans and its complex with a peptide from a phage display library: implications for the identification of peptide mimotopes” J Mol Biol 1997 Dec. 12; 274(4):622-34; Giebel L B et al. “Screening of cyclic peptide phage libraries identifies ligands that bind streptavidin with high affinities” Biochemistry 1995 Nov. 28; 34(47):15430-5; Davies E L et al., “Selection of specific phage-display antibodies using libraries derived from chicken immunoglobulin genes” J Immunol Methods 1995 Oct. 12; 186(1):125-35; Jones C R T al. “Current trends in molecular recognition and bioseparation” J Chromatogr A 1995 Jul. 14; 707(1):3-22; Deng S J et al. “Basis for selection of improved carbohydrate-binding single-chain antibodies from synthetic gene libraries” Proc Natl Acad Sci U S A 1995 May 23; 92(11):4992-6; and Deng S J et al. “Selection of antibody single-chain variable fragments with improved carbohydrate binding by phage display” J Biol Chem 1994 Apr. 1; 269(13):9533-8, which are incorporated herein by reference.

Peptide sequences capable of binding Tsg101 or other biomolecular targets of Ta1 can also be uncovered using computational biology. For example, various peptide sequences derived from Ta1 can be computationally analyzed for an ability to bind Tsg101 or any other molecular target using a variety of three dimensional computational tools. Software programs useful for displaying three-dimensional structural models, such as RIBBONS (Carson, M., 1997. Methods in Enzymology 277, 25), O (Jones, T A. et al., 1991. Acta Crystallogr. A47, 110), DINO (DINO: Visualizing Structural Biology (2001) http://www.dino3d.org); and QUANTA, INSIGHT, SYBYL, MACROMODE, ICM, MOLMOL, RASMOL and GRASP (reviewed in Kraulis, J., 1991. Appl Crystallogr. 24, 946) can be utilized to model interactions between Tsg101 and prospective peptide sequences to thereby identify peptides which display the highest probability of binding to a specific Tsg101 region. Computational modeling of protein-peptide interactions has been successfully used in rational drug design, for further detail, see Lam et al., 1994. Science 263, 380; Wlodawer et al., 1993. Ann Rev Biochem. 62, 543; Appelt, 1993. Perspectives in Drug Discovery and Design 1, 23; Erickson, 1993. Perspectives in Drug Discovery and Design 1, 109, and Mauro M J. et al., 2002. J Clin Oncol. 20, 325-34.

It will be appreciated that peptides identified according to the teachings of the present invention may be degradation products, synthetic peptides or recombinant peptides as well as peptidomimetics, typically, synthetic peptides and peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated bonds (—N(CH3)—CO—), ester bonds (—C(R)H—C—O—O—C(R)—N—), ketomethylen bonds (—CO—CH2-), α-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), peptide derivatives (—N(R)—CH2-CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as Phenylglycine, TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables 1 and 2 below list naturally occurring amino acids (Table 1) and non-conventional or modified amino acids (Table 2) which can be used with the present invention.

TABLE 1

| | Three-Letter | |
|---|---|---|
| Amino Acid | Abbreviation | One-letter Symbol |
| alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| glycine | Gly | G |
| Histidine | His | H |
| isoleucine | Iie | I |
| leucine | Leu | L |
| Lysine | Lys | K |
| Methionine | Met | M |
| phenylalanine | Phe | F |
| Proline | Pro | P |
| Serine | Ser | S |
| Threonine | Thr | T |
| tryptophan | Trp | W |
| tyrosine | Tyr | Y |
| Valine | Val | V |
| Any amino acid as above | Xaa | X |

TABLE 2

| Non-conventional amino acid | Code |
|---|---|
| α-aminobutyric acid | Abu |
| α-amino-α-methylbutyrate | Mgabu |
| aminocyclopropane- | Cpro |
| carboxylate | |
| aminoisobutyric acid | Aib |
| aminonorbornyl- | Norb |
| carboxylate | |
| cyclohexylalanine | Chexa |
| cyclopentylalanine | Cpen |
| D-alanine | Dal |
| D-arginine | Darg |
| D-aspartic acid | Dasp |
| D-cysteine | Dcys |
| D-glutamine | Dgln |
| D-glutamic acid | Dglu |
| D-histidine | Dhis |
| D-isoleucine | Dile |
| D-leucine | Dleu |
| D-lysine | Dlys |
| D-methionine | Dmet |
| D-ornithine | Dorn |
| D-phenylalanine | Dphe |
| D-proline | Dpro |
| D-serine | Dser |
| D-threonine | Dthr |
| D-tryptophan | Dtrp |
| D-tyrosine | Dtyr |
| D-valine | Dval |
| D-α-methylalanine | Dmala |
| D-α-methylarginine | Dmarg |
| D-α-methylasparagine | Dmasn |
| D-α-methylaspartate | Dmasp |
| D-α-methylcysteine | Dmcys |
| D-α-methylglutamine | Dmgln |
| D-α-methylhistidine | Dmhis |
| D-α-methylisoleucine | Dmile |
| D-α-methylleucine | Dmleu |
| D-α-methyllysine | Dmlys |
| D-α-methylmethionine | Dmmet |
| D-α-methylornithine | Dmorn |
| D-α-methylphenylalanine | Dmphe |
| D-α-methylproline | Dmpro |
| D-α-methylserine | Dmser |
| D-α-methylthreonine | Dmthr |
| D-α-methyltryptophan | Dmtrp |
| D-α-methyltyrosine | Dmty |
| D-α-methylvaline | Dmval |
| D-α-methylalnine | Dnmala |
| D-α-methylarginine | Dnmarg |
| D-α-methylasparagine | Dnmasn |
| D-α-methylasparatate | Dnmasp |
| D-α-methylcysteine | Dnmcys |
| D-N-methylleucine | Dnmleu |
| D-N-methyllysine | Dnmlys |
| N-methylcyclohexylalanine | Nmchexa |
| D-N-methylornithine | Dnmorn |
| N-methylglycine | Nala |
| N-methylaminoisobutyrate | Nmaib |
| N-(1-methylpropyl)glycine | Nile |
| N-(2-methylpropyl)glycine | Nile |
| N-(2-methylpropyl)glycine | Nleu |
| D-N-methyltryptophan | Dnmtrp |
| D-N-methyltyrosine | Dnmtyr |
| D-N-methylvaline | Dnmval |
| γ-aminobutyric acid | Gabu |
| L-t-butylglycine | Tbug |
| L-ethylglycine | Etg |
| L-homophenylalanine | Hphe |
| L-α-methylarginine | Marg |
| L-α-methylaspartate | Masp |
| L-α-methylcysteine | Mcys |
| L-α-methylglutamine | Mgln |
| L-α-methylhistidine | Mhis |
| L-α-methylisoleucine | Mile |
| D-N-methylglutamine | Dnmgln |
| D-N-methylglutamate | Dnmglu |
| D-N-methylhistidine | Dnmhis |
| D-N-methylisoleucine | Dnmile |
| D-N-methylleucine | Dnmleu |
| D-N-methyllysine | Dnmlys |
| N-methylcyclohexylalanine | Nmchexa |
| D-N-methylornithine | Dnmorn |
| N-methylglycine | Nala |
| N-methylaminoisobutyrate | Nmaib |
| N-(1-methylpropyl)glycine | Nile |
| N-(2-methylpropyl)glycine | Nleu |
| D-N-methyltryptophan | Dnmtrp |
| D-N-methyltyrosine | Dnmtyr |
| D-N-methylvaline | Dnmval |
| γ-aminobutyric acid | Gabu |
| L-t-butylglycine | Tbug |
| L-ethylglycine | Etg |
| L-homophenylalanine | Hphe |
| L-α-methylarginine | Marg |
| L-α-methylaspartate | Masp |
| L-α-methylcysteine | Mcys |
| L-α-methylglutamine | Mgln |
| L-α-methylhistidine | Mhis |
| L-α-methylisoleucine | Mile |
| L-α-methylleucine | Mleu |
| L-α-methylmethionine | Mmet |
| L-α-methylnorvaline | Mnva |
| L-α-methylphenylalanine | Mphe |
| L-α-methylserine | Mser |
| L-α-methylvaline | Mtrp |
| L-α-methylleucine | Mval Nnbhm |
| N-(N-(2,2-diphenylethyl)carbamylmethyl-glycine | Nnbhm |
| 1-carboxy-1-(2,2-diphenylethylamino)cyclopropane | Nmbc |
| L-N-methylalanine | Nmala |
| L-N-methylarginine | Nmarg |
| L-N-methylasparagine | Nmasn |
| L-N-methylaspartic acid | Nmasp |
| L-N-methylcysteine | Nmcys |
| L-N-methylglutamine | Nmgin |
| L-N-methylglutamic acid | Nmglu |
| L-N-methylhistidine | Nmhis |
| L-N-methylisolleucine | Nmile |
| L-N-methylleucine | Nmleu |
| L-N-methyllysine | Nmlys |
| L-N-methylmethionine | Nmmet |
| L-N-methylnorleucine | Nmnle |
| L-N-methylnorvaline | Nmnva |
| L-N-methylornithine | Nmorn |
| L-N-methylphenylalanine | Nmphe |
| L-N-methylproline | Nmpro |
| L-N-methylserine | Nmser |
| L-N-methylthreonine | Nmthr |
| L-N-methyltryptophan | Nmtrp |
| L-N-methyltyrosine | Nmtyr |
| L-N-methylvaline | Nmval |
| L-N-methylethylglycine | Nmetg |
| L-N-methyl-t-butylglycine | Nmtbug |
| L-norleucine | Nle |
| L-norvaline | Nva |
| α-methyl-aminoisobutyrate | Maib |
| α-methyl-γ-aminobutyrate | Mgabu |
| α-methylcyclohexylalanine | Mchexa |
| α-methylcyclopentylalanine | Mcpen |
| α-methyl-α-napthylalanine | Manap |
| α-methylpenicillamine | Mpen |
| N-(4-aminobutyl)glycine | Nglu |
| N-(2-aminoethyl)glycine | Naeg |
| N-(3-aminopropyl)glycine | Norn |
| N-ammo-α-methylbutyrate | Nmaabu |
| α-napthylalanine | Anap |
| N-benzylglycine | Nphe |
| N-(2-carbamylethyl)glycine | Ngln |
| N-(carbamylmethyl)glycine | Nasn |
| N-(2-carboxyethyl)glycine | Nglu |
| N-(carboxymethyl)glycine | Nasp |
| N-cyclobutylglycine | Ncbut |
| N-cycloheptylglycine | Nchep |
| N-cyclohexylglycine | Nchex |
| N-cyclodecylglycine | Ncdec |
| N-cyclododeclglycine | Ncdod |
| N-cyclooctylglycine | Ncoct |
| N-cyclopropylglycine | Ncpro |
| N-cycloundecylglycine | Ncund |
| N-(2,2-diphenylethyl)glycine | Nbhm |
| N-(3,3-diphenylpropyl)glycine | Nbhe |
| N-(3-indolylyethyl)glycine | Nhtrp |
| N-methyl-γ-aminobutyrate | Nmgabu |
| D-N-methylmethionine | Dnmmet |
| N-methylcyclopentylalanine | Nmcpen |
| D-N-methylphenylalanine | Dnmphe |
| D-N-methylproline | Dnmpro |
| D-N-methylserine | Dnmser |
| D-N-methylserine | Dnmser |
| D-N-methylthreonine | Dnmthr |
| N-(1-methylethyl)glycine | Nva |
| N-methyla-napthylalanine | Nmanap |
| N-methylpenicillamine | Nmpen |
| N-(p-hydroxyphenyl)glycine | Nhtyr |
| N-(thiomethyl)glycine | Ncys |
| penicillamine | Pen |
| L-α-methylalanine | Mala |
| L-α-methylasparagine | Masn |
| L-α-methyl-t-butylglycine | Mtbug |
| L-methylethylglycine | Metg |
| L-α-methylglutamate | Mglu |
| L-α-methylhomophenylalanine | Mhphe |
| N-(2-methylthioethyl)glycine | Nmet |
| N-(3-guanidinopropyl)glycine | Narg |
| N-(1-hydroxyethyl)glycine | Nthr |
| N-(hydroxyethyl)glycine | Nser |
| N-(imidazolylethyl)glycine | Nhis |
| N-(3-indolylyethyl)glycine | Nhtrp |
| N-methyl-γ-aminobutyrate | Nmgabu |
| D-N-memylmethionine | Dnmmet |
| N-methylcyclopentylalanine | Nmcpen |
| D-N-methylphenylalanine | Dnmphe |
| D-N-methylproline | Dnmpro |
| D-N-methylserine | Dnmser |
| D-N-methylthreonine | Dnmthr |
| N-(1-methylethyl)glycine | Nval |
| N-methyla-napthylalanine | Nmanap |
| N-methylpenicillamine | Nmpen |
| N-(p-hydroxyphenyl)glycine | Nhtyr |
| N-(thiomethyl)glycine | Ncys |
| penicillamine | Pen |
| L-α-methylalanine | Mala |
| L-α-methylasparagine | Masn |
| L-α-methyl-t-butylglycine | Mtbug |
| L-methylethylglycine | Metg |
| L-α-methylglutamate | Mglu |
| L-α-methylhomophenylalanine | Mhphe |
| N-(2-methylthioethyl)glycine | Nmet |
| L-α-methyllysine | Mlys |
| L-α-methylnorleucine | Mnle |
| L-α-methylornithine | Morn |
| L-α-methylproline | Mpro |
| L-α-methylthreonine | Mthr |
| L-α-methyltyrosine | Mtyr |
| L-N-methylhomophenylalanine | Nmhphe |
| N-(N-(3,3-diphenylpropyl)carbamylmethyl(1)glycine | Nnbhe |

Since the peptides of the present invention are preferably utilized in therapeutics which require the peptides to be in soluble form, the peptides of the present invention preferably include one or more non-natural or natural polar amino acids, including but not limited to serine and threonine which are capable of increasing peptide solubility due to their hydroxyl-containing side chain.

The peptides of the present invention are preferably utilized in a linear form, although it will be appreciated that in cases where cyclicization does not severely interfere with peptide characteristics, cyclic forms of the peptide can also be utilized.

The peptides of present invention can be biochemically synthesized such as by using standard solid phase techniques. These methods include exclusive solid phase synthesis, partial solid phase synthesis methods, fragment condensation, classical solution synthesis. These methods are preferably used when the peptide is relatively short (i.e., 10 kDa) and/or When it cannot be produced by recombinant techniques (i.e., not encoded by a nucleic acid sequence) and therefore involves different chemistry.

Solid phase peptide synthesis procedures are well known in the art and further described by John Morrow Stewart and Janis Dillaha Young, Solid Phase Peptide Syntheses (2nd Ed., Pierce Chemical Company, 1984).

Synthetic peptides can be purified by preparative high performance liquid chromatography [Creighton T. (1983) Proteins, structures and molecular principles. WH Freeman and Co. N.Y.] and the composition of which can be confirmed via amino acid sequencing.

In cases where large amounts of the peptides of the present invention are desired, the peptides of the present invention can be generated using recombinant techniques such as described by Bitter et al., (1987) Methods in Enzymol. 153:516-544, Studier et al. (1990) Methods in Enzymol. 185:60-89, Brisson et al. (1984) Nature 310:511-514, Takamatsu et al. (1987) EMBO J. 6:307-311, Coruzzi et al. (1984) EMBO J. 3:1671-1680 and Brogli et al., (1984) Science 224:838-843, Gurley et al. (1986) Mol. Cell. Biol. 6:559-565 and Weissbach & Weissbach, 1988, Methods for Plant Molecular Biology, Academic Press, NY, Section VIII, pp 421-463.

As mentioned hereinabove, newly isolated Ta1 and compositions derived therefrom (e.g., peptides) can be used to treat HIV (or SIV) infection, since as shown in the Examples section over expression of Ta1 partially inhibits viral budding.

Thus, according to another aspect of the present invention there is provided a method of treating AIDS in a subject.

As mentioned hereinabove, a subject according to the present invention is a mammal, preferably a human which is infected with the aids virus or is at risk of being infected with the aids virus.

The term “treating” refers to alleviating or diminishing a symptom associated with HIV infection. Preferably, treating cures, e.g., substantially eliminates, the symptoms associated with the infection and/or substantially decreases the viral load in the infected tissue.

The method is effected by providing to the subject a therapeutically effective amount of a Ta1 polypeptide (i.e., full length protein of fragments thereof such as described hereinabove) being at least 80%, at least 85%, at least 90%, at least 91%, at least 92% or more, say 95%-100% homologous to SEQ ID NO: 2, as determined using the BestFit software of the Wisconsin sequence analysis package, utilizing the Smith and Waterman algorithm, where the gap creation equals 8 and gap extension penalty equals 2 or an active portion thereof, as described above, to thereby treat the HIV infection in the subject.

Alternatively, the method is effected by providing a Ta1 polypeptide (i.e., full length protein of fragments thereof such as described hereinabove) being at least 80%, at least 85%, at least 90%, at least 91%, at least 92% or more, say 95%-100% identical to SEQ ID NO: 2, as determined using identical to SEQ ID NO: 2, as determined using the BestFit software of the Wisconsin sequence analysis package, utilizing the Smith and Waterman algorithm, where gap weight equals 50, length weight equals 3, average match equals 10 and average mismatch equals −9.

Provision can be effected by administering the polypeptide or peptide of the present invention to the subject per se, or as part of a pharmaceutical composition where it is mixed with a pharmaceutically acceptable carrier.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the polypeptide, polynucleotide or peptide, such as described hereinabove, which is accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases. One of the ingredients included in the pharmaceutically acceptable carrier can be for example polyethylene glycol (PEG), a biocompatible polymer with a wide range of solubility in both organic and aqueous media (Mutter et al. (1979)).

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, intraperitoneal, intranasal, or intraocular injections.

Alternately, one may administer a preparation in a local rather than systemic manner, for example, via injection of the preparation directly into a specific region of a patient's body.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the invention may be formulated in aqueous solutions, preferably in physiologically compatible buffers such as Hank's solution, Ringer's solution, or physiological salt buffer. For transmucosal administration, penetrants appropriate to the barrier to be permeated are used in the formulation Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The preparations described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The preparation of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients effective to prevent, alleviate or ameliorate symptoms of disease or prolong the survival of the subject being treated.

Determination of a therapeutically effective amount is well within the capability of those skilled in the art.

For any preparation used in the methods of the invention, the therapeutically effective amount or dose can be estimated initially from in vitro assays. For example, a dose can be formulated in animal models and such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. [See e.g., Fingl, et al., (1975) “The Pharmacological Basis of Therapeutics”, Ch. 1 p. 1].

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions including the preparation of the present invention formulated in a compatible pharmaceutical carrier may also be prepared, placed in an appropriate container, and labeled for treatment of an indicated condition.

Compositions of the present invention may, if desired, be presented in a pack or dispenser device, such as an FDA approved kit, which may contain one or more unit dosage forms containing the active ingredient. In addition, other additives such as stabilizers, buffers, blockers and the like may also be added.

It will be appreciated that the compositions of the present invention can be packaged in a one or more containers with appropriate buffers and preservatives and used for therapeutic treatment.

The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

It will be appreciated that the polypeptides or active portions thereof (e.g., peptides) of the present invention can also be provided to the subject by administering to the subject an expressible nucleic acid construct including a polynucleotide being at least 75%, at least 80%, at least 85%, at least 90%, at least 915%, at least 92% or more, say 95%-100% identical to SEQ ID NO: 1 as determined using as determined using the BestFit software of the Wisconsin sequence analysis package, utilizing the Smith and Waterman algorithm, where gap weight equals 50, length weight equals 3, average match equals 10 and average mismatch equals −9 or active portions thereof (i.e., in-vivo gene therapy), or by administering cells transformed with the expressible nucleic acid construct(i.e., ex-vivo gene therapy).

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide or antibody from a host cell in which it is placed. Preferably the signal sequence for this purpose is a mammalian signal sequence. Optionally, the construct may also include a signal that directs polyadenylation, as well as one or more restriction sites and a translation termination sequence. For example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

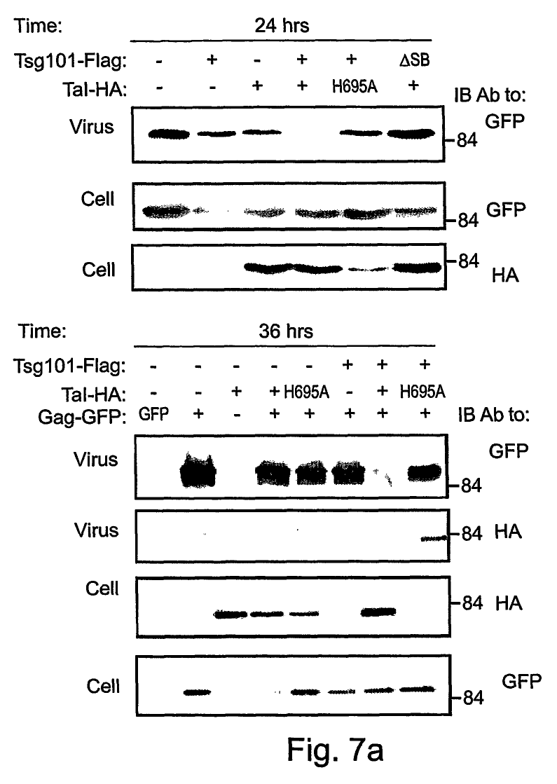

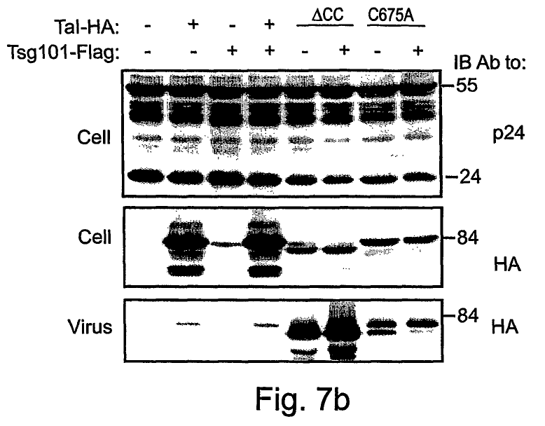

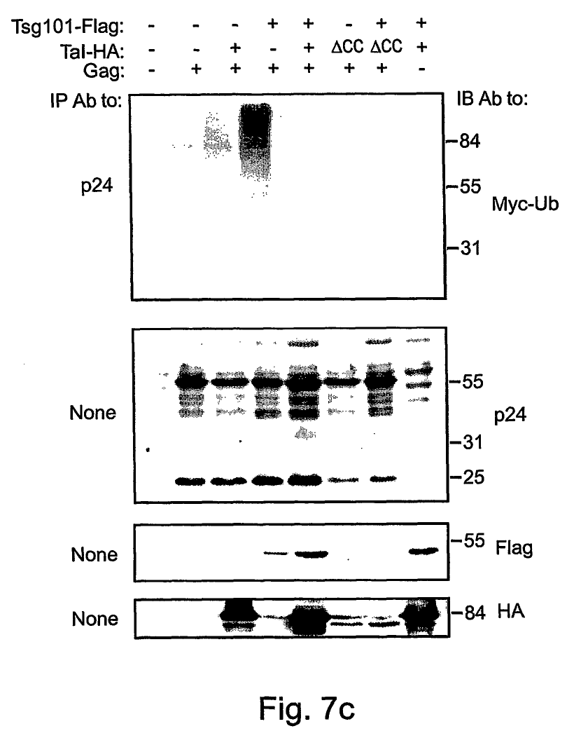

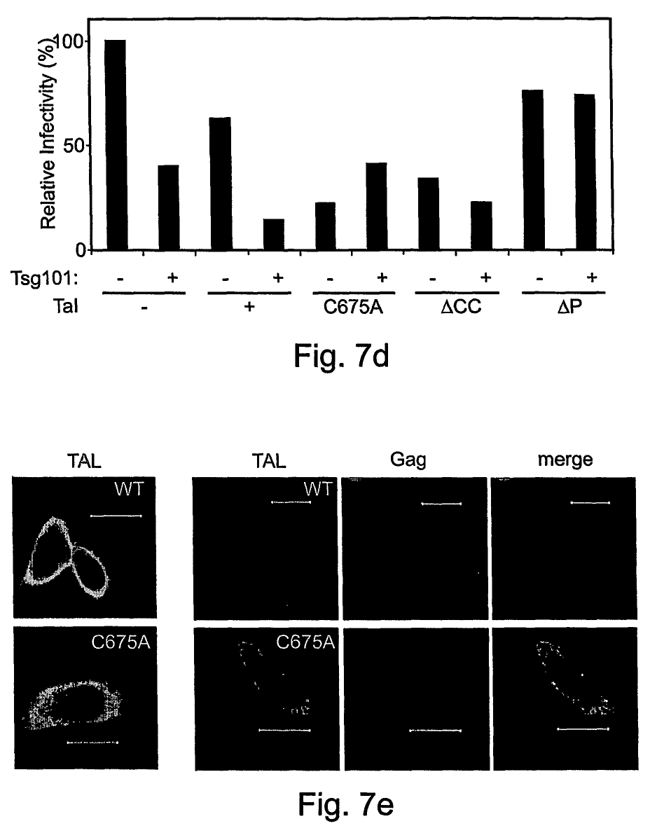

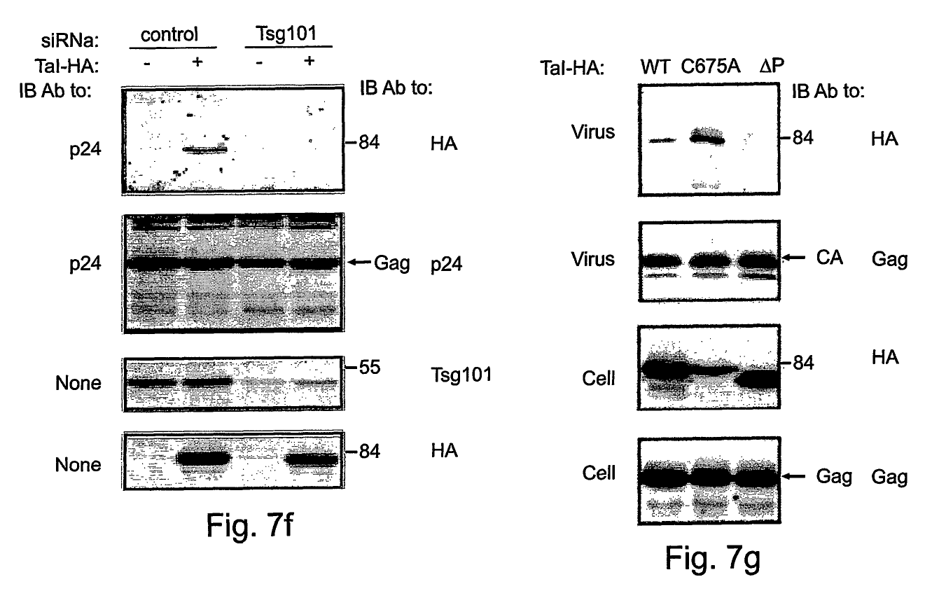

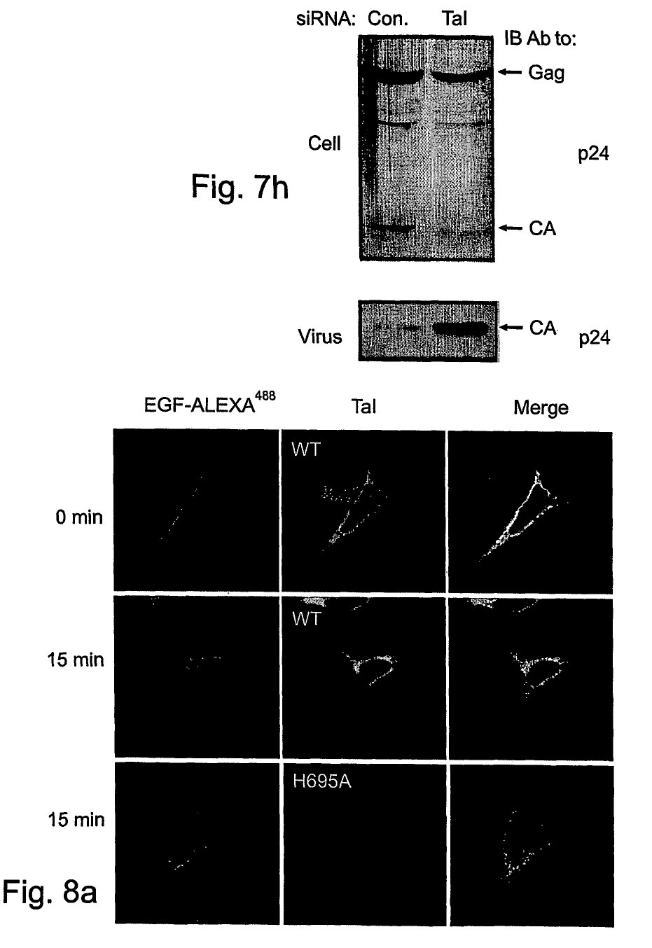

While reducing the present invention to practice, the present inventors demonstrated that inactive mutants of Ta1 are capable of significantly reducing HIV infectivity, a process which requires not only budding but also correct virus processing and maturation. As is shown in Example 6 of the Examples section which follows, RING-finger or coiled-coil mutants of Ta1 are very potent inhibitors of HIV infectivity (e.g., see FIG. 7d). These findings suggest that down-regulation of Ta1 can be used to treat HIV infection.

Down regulation of Ta1 may be effected by an agent capable of downregulating transcription, translation or activity of Ta1.

One example, of an agent capable of downregulating Ta1 activity is an antibody or antibody fragment capable of specifically binding the RING finger domain which is essential for Tal's E3 activity. Preferably, the antibody specifically binds at least one epitope of the RING finger domain. As used herein, the term “epitope” refers to any antigenic determinant on an antigen to which the paratope of an antibody binds.

Epitopic determinants usually consist of chemically active surface groupings of molecules such as amino acids or carbohydrate side chains and usually have specific three dimensional structural characteristics, as well as specific charge characteristics.

The term “antibody” as used in this invention includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polygonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (197201]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fe), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous imbunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10,: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Another agent capable of downregulating Ta1 is a small interfering RNA (siRNA) molecule. RNA interference is a two step process. the first step, which is termed as the initiation step, input dsRNA is digested into 21-23 nucleotide (nt) small interfering RNAs (siRNA), probably by the action of Dicer, a member of the RNase III family of dsRNA-specific ribonucleases, which processes (cleaves) dsRNA (introduced directly or via a transgene or a virus) in an ATP-dependent manner. Successive cleavage events degrade the RNA to 19-21 bp duplexes (siRNA), each with 2-nucleotide 3′ overhangs [Hutvagner and Zamore Curr. Opin. Genetics and Development 12:225-232 (2002); and Bernstein Nature 409:363-366 (2001)].

In the effector step, the siRNA duplexes bind to a nuclease complex to from the RNA-induced silencing complex (RISC). An ATP-dependent unwinding of the siRNA duplex is required for activation of the RISC. The active RISC then targets the homologous transcript by base pairing interactions and cleaves the mRNA into 12 nucleotide fragments from the 3′ terminus of the siRNA [Hutvagner and Zamore Curr. Opin. Genetics and Development 12:225-232 (2002); Hammond et al. (2001) Nat. Rev. Gen. 2:110-119 (2001); and Sharp Genes. Dev. 15:485-90 (2001)]. Although the mechanism of cleavage is still to be elucidated, research indicates that each RISC contains a single siRNA and an RNase [Hutvagner and Zamore Curr. Opin. Genetics and Development 12:225-232 (2002)].

Because of the remarkable potency of RNAi, an amplification step within the RNAi pathway has been suggested. Amplification could occur by copying of the input dsRNAs which would generate more siRNAs, or by replication of the siRNAs formed. Alternatively or additionally, amplification could be effected by multiple turnover events of the RISC [Hammond et al. Nat. Rev. Gen. 2:110-119 (2001), Sharp Genes. Dev. 15:485-90 (2001); Hutvagner and Zamore Curr. Opin. Genetics and Development 12:225-232]. (2002)]. For more information on RNAi see the following reviews Tuschl ChemBiochem. 2:239-245 (2001); Cullen Nat. Immunol. 3:597-599 (2002); and Brantl Biochem. Biophys. Act 1575:15-25 (2002).

Synthesis of RNAi molecules suitable for use with the present invention can be effected as follows. First, the Ta1 mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www.ambiono.com/techlib/tn/91/912. html).

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

Examples of hTa1 specific siRNA sequences, which are effective in down-regulating hTa1 are set forth in SEQ ID NOs: 45-48 (see FIG. 7h and Example 6 of the Examples section which follows).

Another agent capable of downregulating Ta1 is a DNAzyme molecule capable of specifically cleaving an mRNA transcript or DNA sequence of Ta1. DNAzymes are single-stranded polynucleotides which are capable of cleaving both single and double stranded target sequences (Breaker, R. R. and Joyce, G. Chemistry and Biology 1995;2:655; Santoro, S. W. & Joyce, G. F. Proc. Natl, Acad. Sci. USA 1997;943:4262) A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, LM [Curr Opin Mol Ther 4:119-21 (2002)].

Examples of construction and amplification of synthetic, engineered DNAzymes recognizing single and double-stranded target cleavage sites have been disclosed in U.S. Pat No. 6,326,174 to Joyce et al. DNAzymes of similar design directed against the human Urokinase receptor were recently observed to inhibit Urokinase receptor expression, and successfully inhibit colon cancer cell metastasis in vivo (Itoh et al , 20002, Abstract 409, Ann Meeting Am Soc Gen Ther www.asgt.org). In another application, DNAzymes complementary to bcr-ab1 oncogenes were successful in inhibiting the oncogenes expression in-leukemia cells, and lessening relapse rates in autologous bone marrow transplant in cases of CML and ALL.

Downregulation of Ta1 can also be effected by using an antisense polynucleotide capable of specifically hybridizing with an mRNA transcript encoding the Ta1 polypeptide.

Design of antisense molecules which can be used to efficiently downregulate Ta1 must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Luft J Mol Med 76: 75-6 (1998); Kronenwett et al. Blood 91: 852-62 (1998); Rajur et al. Bioconjug Chem 8: 935-40 (1997); Lavigne et al. Biochem Biophys Res Commun 237: 566-71 (1997) and Aoki et al. (1997) Biochem Biophys Res Commun 231: 540-5 (1997)].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in vitro system were also published (Matveeva et al., Nature Biotechnology 16: 1374-1375 (1998)].

Several clinical trials have demonstrated safety, feasibility and activity of antisense oligonucleotides. For example, antisense oligonucleotides suitable for the treatment of cancer have been successfully used [Holmund et al., Curr Opin Mol Ther 1:372-85 (1999)], while treatment of hematological malignancies via antisense oligonucleotides targeting c-myb gene, p53 and Bcl-2 had entered clinical trials and had been shown to be tolerated by patients [Gerwitz Curr Opin Mol Ther 1:297-306 is (1999)].

More recently, antisense-mediated suppression of human heparanase gene expression has been reported to inhibit pleural dissemination of human cancer cells in a mouse model [Uno et al., Cancer Res 61:7855-60 (2001)].

Thus, the current consensus is that recent developments in the field of antisense technology which, as described above, have led to the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

Another agent capable of downregulating Ta1 is a ribozyme molecule capable of specifically cleaving an mRNA transcript encoding a Ta1 polypeptide. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic applications. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—WEB home page).

Oligonucleotide agents utilized by the present invention can be generated according to any oligonucleotide synthesis method known in the art such as enzymatic synthesis or solid phase synthesis. Equipment and reagents for executing solid-phase synthesis are commercially available from, for example, Applied Biosystems. Any other means for such synthesis may also be employed; the actual synthesis of the oligonucleotides is well within the capabilities of one skilled in the art and. can be accomplished via established methodologies as detailed in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988) and “Oligonucleotide Synthesis” Gait, M. J., ed. (1984) utilizing solid phase chemistry, e.g. cyanoethyl phosphoramidite followed by deprotection, desalting and purification by for example, an automated trityl-on method or HPLC.

The oligonucleotide of the present invention is of at least 17, at least 18, at feast 19, at least 20, at least 22, at least 25, at least 30 or at least 40, bases according to the function thereof. Thus, for example, oligonucleotides of a small interfering duplex oligonucleotide are preferably 21-23 bases long.

The oligonucleotides of the present invention may comprise heterocylic nucleosides consisting of purines and the pyrimidines bases, bonded in a 3′ to 5′ phosphodiester linkage.

Preferably used oligonucleotides are those modified in either backbone, internucleoside linkages or bases, as is broadly described hereinunder. Such modifications can oftentimes facilitate oligonucleotide uptake and resistivity to intracellular conditions.

Specific examples of preferred oligonucleotides useful according to this aspect of the present, invention include oligonucleotides containing modified backbones or non-natural internucleoside linkages. Oligonucleotides having modified backbones include those that retain a phosphorus atom in the backbone, as disclosed in U.S. Pat. Nos: 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466, 677; 5,476,925; 5,519,126; 5,536,821; 5,541,306; 5,550,111; 5,563,253; 5,571,799; 5,587,361; and 5,625,050.

Preferred modified oligonucleotide backbones include, for example, phosphorothioates, chiral phosphorothioates, phosphorodithioates, phosphotriesters, aminoalkyl phosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms can also be used.

Alternatively, modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts, as disclosed in U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623, 070; 5,663,312; 5,633,360; 5,677,437; and 5,677,439.

Other oligonucleotides which can be used according to the present invention, are those modified in both sugar and the internucleoside linkage, i.e., the backbone, of the nucleotide units are replaced with novel groups. The base units are maintained for complementation with the appropriate polynucleotide target. An example for such an oligonucleotide mimetic, includes peptide nucleic acid (PNA). A PNA oligonucleotide refers to an oligonucleotide where the sugar-backbone is replaced with an amide containing backbone, in particular an aminoethylglycine backbone. The bases are retained and are bound directly or indirectly to aza nitrogen atoms of the amide portion of the backbone. United States patents that teach the preparation of PNA compounds include, but are not limited to, U.S. Pat. Nos. 5,539,082; 5,714,331; and 5,719,262, each of which is herein incorporated by reference. Other backbone modifications, which can be used in the present invention are disclosed in U.S. Pat. No: 6,303,374.

Oligonucleotides of the present invention may also include base modifications or substitutions. As used herein, “unmodified” or “natural” bases include the purine bases adenine (A) and guanine (G), and the pyrimidine bases thymine (T), cytosine (C) and uracil (U). Modified bases include but are not limited to other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further bases include those disclosed in U.S. Pat. No: 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Such bases are particularly useful for increasing the binding affinity of the oligomeric compounds of the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. [Sanghvi YS et al. (1993) Antisense Research and Applications, CRC Press, Boca Raton 276-278] and are presently preferred base substitutions, even more particularly when combined with 2′-O-methoxyethyl sugar modifications.

It is not necessary for all positions in a given oligonucleotide molecule to be uniformly modified, and in fact more than one of the aforementioned modifications may be incorporated in a single compound or even at a single nucleoside within an oligonucleotide.

It will be appreciated that small molecule agents directed at the RING finger domain of Ta1 may also be employed to downregulate the activity of Ta1. Such compounds were reported to block the ligase activity of another RING-bearing ligase, essentially Hdm2 (Z. Lai et al. PNAS vol. 99, pp 14734-14739, 2002).

The above-described methodology may be used to treat other retrovirus-associated diseases since it is well established that all retroviruses funnel into the Vps pathway for egress from the cell and Tsg101 has been implicated in the budding process of enveloped viruses [reviewed in Pornillos (2002b) Trends Cell Biol. 12:569-79].

Thus, it is conceivable that Ta1, or active portions thereof, can be used to inhibit budding of retroviruses other than HIV possibly by interfering with the function (i.e., stabilization or disruption) of oligomeric complexes (e.g., ESCRT-1) known to be essential for the viral budding process [see Garrus (2001) Cell 107:55-65; and Martin-Serrano (2003) J. Virol. 77:1313-9].

Prior art studies have established that cells either overexpressing or lacking expression of Tsg101 exhibit defects in endosomal trafficking which leads to prolonged-signaling from cell-surface molecules and results in enhanced cell proliferation which ultimately leads to tumorogenesis [Babst (2000) Traffic 1:248-58].

It is notable that Tsg101 participates in tumorogenesis. For example, it was shown that functional inactivation of tsg101 in mouse NIH3T3 fibroblasts leads to cellular transformation, and the transformed cells can form metastatic tumors in nude mice. The neoplastic transformation and tumorogenesis are reversible by restoration of tsg101 function. [Li and cohen (1996) Cell 85:319-329]. Tsg101 has been mapped to chromosome 11p, bands 15.1-15.2 [Li et al (1997) Cell 10;88(1):143-54] a region known to exhibit loss of heterozygosity in a variety of human malignancies [Weitzel et al (1994) Gynecol Oncol. 55(2):245-52], suggesting that Tsg101 might be a Tumor suppressor. Although no genomic deletion has been identified, aberrant transcripts can be found in various tumors [Carney et al (1998) J Soc Gynecol Investig. 5(5):281-5; Chang et al (1999) Br J Cancer. 79(3-4):445-50; Wang et al (2000) Oncogene 16:677-9]. Abnormally spliced transcripts of Tsg101 have been found to very closely correlate with tumor grades and p53 mutations in breast cancer samples. Stress conditions such as hypoxia induce splicing transcripts in primary lymphocytes Turpin et al 1999).

Thus, the present invention also contemplates use of the present therapeutic agents in treatment of tumorous diseases, such as cancer. Such treatment can be effected by systemic or intra-tumor administration of the oligonucleotides, polypeptides (e.g., see Example 7 of the Examples section) and antibodies described hereinabove and monitoring of tumor progression until satisfactory tumor reduction is achieved and tumorogenesis is halted.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes”IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

Materials and Experimental Procedures

Materials

Chemicals—The composition of buffers was previously described [Waterman et al., (1999) J Biol Chem 274, 22151-22154]. Unless otherwise indicated all chemicals were purchased from Sigma-Aldrich St. Louis Mo. USA. Missouri Yeast growth media was purchased from Becton Dickenson Sparks. Human recombinant EGF was purchased from Sigma (St. Louis, Mo.). Na125I (1000 mCi) was purchased from Amersham Pharmacia. Biotech (Buckinghamshire, UK). IODOGEN was from Pierce (Rochford, Ill.). A protease inhibitor cocktail (set III) was from Calbiochem (San Diego, Calif.).

Tissue culture reagents—Fetal calf serum, and L-Glutamine were purchased from Biological Industries (Beit Haemek, Israel). Dulbecco's Modified Eagle's Medium (DMEM), DMEM:F12 (1:1), F12, Lipofectamine, and a penicillin-streptomycin mixture were supplied by Gibco BRL (Grand Island, N.Y.).

Antibodies—An anti-hemagglutinin (HA) mAb was purchased from Boehringer Mannheim. A mAb to the active doubly phosphorylated form of Erk was from Sigma (Anti DP-Erk, Cat. No. M8159, Sigma-Aldrich St. Louis Mo. USA). An anti hemagglutinin (HA) rat monoclonal (mAb) antibody 3F10 was purchased from Roche Molecular Biochemicals (Mannheim, Germany), and an anti Flag mouse mAb was from Sigma (St. Louis, Mo. USA). Murine mAb SG565 to the EGFR was generated in mice immunized with a recombinant extracellular portion of the human EGFR. A polyclonal antiserum for immunoblot analysis of EGFR was from Santa Cruz Biotechnology (Santa Cruz, Calif.). Murine monoclonal IgG (C-2) and goat polyclonal (M-19) anti Tsg101 antibodies, anti c-Myc mAb, and rabbit polyclonal anti GST antibody were also from Santa Cruz Biotechnology. An anti GFP mAb was purchased from BD Biosciences Clontech (Palo Alto, Calif. USA). A goat anti rat peroxidase- conjugated IgG, a goat anti mouse peroxidase- conjugated IgG, donkey anti goat peroxidase- conjugated IgG, a donkey anti rat Cy3- conjugated, and a donkey anti mouse Cy2- conjugated were purchased from Jackson ImmunoResearch (West Grove, Pa). A peroxidase- conjugated protein-A was from ICN (Costa Mesa, Calif.).

Polyacrylamide gel electrophoresis, immunoblot, and immunoprecipitation reagents—Acrylamide (30%/o), ammonium persulfate, and TEMED were from Bio-Rad (Richmond, Calif.). Molecular weight standard proteins were purchased from Sigma-Aldrich (St. Louis, Mo., USA). The ECL chemiluminescence kit used for immunobloting was obtained from Amersham (Buckinghamshire,UK). Nitrocellulose membranes were purchased from Schleicher & Schuell (Dassel, Germany). Anti-mouse IgG-, anti rat IgG-coupled agarose beads, murine anti Flag IgG-conjugated agarose beads, and glutathione-agarose beads were from Sigma Aldrich (St. Louis, Mo., USA).

Molecular biology reagents—Restriction enzymes were purchased from New England Biolabs (Berely, Mass). Extended high fidelity DNA polymerase, and dNTP's were from Roche Molecular Biochemicals (Mannheim, Germany). T4 DNA ligase, and T4 polynucleotide kinase (PNK) were from MBI Farmentase (Vilinius, Lithuania). A pfu turbo DNA polymerase, and a mutagenesis kit were purchased from Stratagene (Cedar Creek, Tex.).

Experimental Procedures

Vectors for two hybrid screening—Yeast expression plasmid pBTM116-LexA (bait), carrying TRP1 selection marker was cut using EcoRI and SalI restriction enzymes. Human Tsg101 coding sequence (GenBank Accession No: BC009239) was PCR amplified from pEFIα/Tsg101 wt using primers: seTsgRI (5′-GGAATTCGTCATGGCGGTGTCGGAG-3′, SEQ ED NO: 9), and as TsgXho (5′-CCTCGAGTCAGTAGAG GTCACTGAGACCG-3′, SEQ ID NO: 10). The resultant PCR product was digested with EcoRI and XhoI, and cloned into EcoRI and SalI sites of pBTM116 to generate pBTM116-Tsg101.

Tsg101 C-terminus (nucleotides 500 to 1290) was PCR amplified using Ubc5RI (5′-GGAATTCGGGCTTATTCAGGTCATGATTG T-3′, SEQ ID NO: 11), and asTsgXho (SEQ ID NO: 10), and cloned as an EcoRI-XhoI fragment into EcoRI and SalI sites in pBTM116 to generate pBTM116/Tsg101 ΔN.

The SB subdomain of Tsg101 (i.e., nucleotides 1041 to 1291) was PCR amplified using BamHISB5 (5′-CCGGGACATTCCCACAGCTCCCTTA TA-3′, SEQ ID-NO: 12), and asTsgXho (SEQ ID NO: 10), and cloned as an BamHI-XhoI fragment into the BamHI and SalI sites of pBTM116, to generate pBTM116/SB.

Tsg101ΔSB was generated using the seTsg101 EcoRI 5′primer (SEQ ID NO: 9) along with a 3′primer AAACTGCAGCCAGAGCAGAACTGAGTTCfTCATCC (SEQ ID NO: 13) containing a PSTI site. The resultant PCR product was cut with EcoRi and PSTI and ligated to these sites in the pBTM116 vector.

Tsg101ΔC was generated using the seTsg101 EcoRI 5′ (SEQ ID NO: 9) primer along with a 3′ primer AAACTGCAGGGCACGATCCATTTCCTC containing a PSTI site (SEQ ID NO: 14). The resultant PCR product was cut with EcoRi and PSTI and ligated to these sites in the pBTM116 vector.

Ta1 CC was generated by cutting the rescued Ta1-pGAD10 from the yeast two hybrid with EcoRI and PSTI and ligating the construct to a pGAD424 (CLONTECH Palo Alto Calif.).

Ta1 ΔPTAP/PSAP was generated using the Forward primer CCTGCAGAGCTGGAGGTGC (SEQ ID NO: 15) and the Reverse primer GACGACCTCACCCATTGGTG (SEQ ID NO: 16) thereby deleting nucleotides 2028-2078 of hTa1.

Construction of mammalian expression vectors—Flag-tagged murine Tsg101 cDNA (provided by S. Cohen INFOsncohen@stanford.edu, Department of Genetics, Stanford University, SUMC L-312A, Mail Code 5120, Stanford Calif. 94305-5120) was amplified by PCR and cloned into the pEF1α mammalian expression vector [Grammatikakis et al., (1999) Mol Cell Biol 19, 1661-72]. Deletion mutants were generated by PCR using the pfu-turbo enzyme (Stratagene, Cedar Creek, Tex.), and primers complementary to regions downstream and upstream to the region to be deleted. Resultant PCR products were subsequently phosphorylated using T4 polynucleotide kinase and ligated.

The following deletion mutants were generated:

ΔUEV-Tsg101 (deletion of nucleotides 259 to 434)-pEF1α/Tsg101 ΔUEV (nucleotides 259 to 434) was generated using UEVR primer (5′-GTATGTATTACCTCTA TAAGGCAC-3′, SEQ ID NO: 17), and UEVF (5′-GGGCTTATTCAGGT CATGATTGT-3′, SEQ ID NO: 18).

ΔPro-Tsg101 (deletion of nucleotides 459 to 725)—pEF1α/Tsg101ΔPro (nucleotides 459 to 725) was generated using primers: ProR (5′-CACAATCATGACCTGAATAAGCC-3′, SEQ ID NO: 19), and ProF (5′-GAGGACACCATCCGA GCCTC-3′, SEQ ID NO: 20).

ΔCC-Tsg101 (deletion of nucleotides 725 to 1010)-pEF1α/Tsg101ΔCC (nucleotides 725 to 1010) was generated using primers: CCR (5′-GAGGCTCGGATGGTGTCCTC-3′, SEQ ID NO: 21), and CCF (5′-CATTCCCACAGCTCCCTTATAC-3′, SEQ ID NO: 22).

ΔSB-Tsg101 (deletion of nucleotides 1031 to 1230)-pEF1α/Tsg101ΔSB (nucleotides 1031 to 1230) was generated using primers: SBR (5′-GTATAAGGGAGCTGTGGGAATG-3′, SEQ ID NO: 23), and SBF (5′-GGAGGTGGAGACTACAAGGAC-3′, SEQ ID NO: 24).

A vector encoding a fusion protein including full-length Tsg101 fused to mGST, was generated by PCR amplification of Tsg101 sequence from pEF1α/Tsg101 using primers: BamHITsg (5′-CCGGGATCCATGGCGGTGTCGGAG-3′, SEQ ID NO: 25), and TsgNotI (5′-ATAGTTTAGCGGCCGCTAGTCACTTGTCATCGTCGTCC-3′, SEQ ID NO: 26). The resulting PCR product was digested and cloned into the BamHI and NotI sites of the mGST expression vector.

hTa1 identified by yeast two hybrid screening was cloned from a cDNA library generated from T47D cells. BC009239 (hTa1, SEQ ID NO: 1) coding sequence was PCR amplified using the following primers: Ta15 ′(5′-CCCAAGCTTGGAAGGATGCCGCTCTT-3′, SEQ ID NO: 27), which contains a HindIII site and Ta13′(5′GGGGTACCCCTCATCAGGCATAATCGGGTACATCATAGGGATAGCTGCTGTGGTAGATGCG-3′, SEQ ID NO: 28), which contains the HA-tag coding sequence and a KpnI site. The resultant PCR product was digested with HindIII and KpnI, and cloned into the HindIII and KpnI sites of the mammalian expression vector pcDNA3.1 (Invitrogen, Rhenium Ltd. Israel). pcDNA3.1/Ta1-HA was used as a template for the following deletion and point mutants:

ΔSAM-Ta1 (deletion of nucleotides 1704 to 1896) was generated by PCR using primers: SAMR (5′-CTCTTCTrGCAGCTTCAAGG-3′, SEQ ID NO: 29), and SAMF (5′-GCCAGGATCCAGCCAGAG-3′, SEQ ID NO: 30).

The T H695A al point mutation was generated using site-directed mutagenesis of pcDNA3.1/Ta1-HA using the following primers: seTa1H695A (5′-CCTCAACTGTGGCGCCGTCTGCTGCTGCC-3′, SEQ ID NO: 31), and asTa1H695A (5′-GGCAGCAGCAGACGGCGCCACAGTTGAGG-3′, SEQ ID NO: 32). All the mutations and deletions were confirmed by DNA sequencing.

Ta1 ΔPTAP/PSAP was generated using the Forward primer 5′-CCTGCAGAGCTGGAGGTGC-3′(SEQ ID NO: 33) and the Reverse primer 5′-GACGACCTCACCCATTGGTG-3′(SEQ ID NO: 34), thereby deleting nucleotides 2028-2078 of hTa1.

Ta1ΔCC was generated using the forward primer 5′-GAGGAGCTGTCGGCTGAGC-3′(SEQ ID NO: 35) and the reverse primer 5′-TAACTTAATCTGGCTCCTGATCTGCCG-3′(SEQ ID NO: 36) thereby deleting nucleotides1518-1820 of hTa1. pGAG-GFP including the rev-independent HIV-1_{HXB2} Gag sequence fused to EGFP was provided by M. Resh [Hermida-Matsumoto and Resh, (2000) J Virol 74, 8670-9].

c-Myc peptide-tagged ubiquitin (Myc-Ub) was constructed by fusion of three copies of c-Myc peptide upstream to a ubiquitin coding sequence in pcDNA3. The expression vector (pcDNA3) encoding human EGFR was described previously [Tzahar et al., (1996) Mol Cell Biol 16, 5276-5287].

Yeast-two hybrid assay—The fill length coding sequence of Tsg101 (GenBank Accession No. NM_{—}006292.) was fused to the LexA DNA binding domain (amino acids 1 to 211) of the pBTM116 bait vector (Constructed from CLONTECH pGBT9 by Fields s and Bartel Proc Natl Acad Sci U S A. 1993 Oct. 1;90(19):9186-90). The L40 yeast strain (Invitrogen, Corp. Rhenium Ltd. Israel) was first transformed with pBTM116-Tsg101, tested for auto-activation and then transformed with a human brain cDNA library (CLONTECH, Palo alto, Calif.) cloned in pGAD10 vector. All transformations were performed using the lithium acetate method as described in CLONTECH YEAST protocol hand book (http://www.clontechcom/techinfo/manuals/PDF/PT3024-1.pdf). Co-transformants were plated onto Trp-Leu-His selective medium supplemented with 3-aminotriazole (5 Mm Sigma St. Louis, Mo.). His⁺ colonies were then assayed for β-galactosidase using a filter lift assay as described in CLONTECH YEAST protocol hand book (http://www.clontech.com/techinfo/manuals/PDF/PT3024-1.pdf). Positive clones were rescued into bacteria and re-transformed into the L40 yeast strain to confirm interactions. Clones confirmed in this manner were sequenced using the 5′pGAD10 sequence amplifier (CLONTECH, Palo Alto, Calif.).

Liquid culture β-galadosidase assays—Naïve L40 yeast cells were transformed with bait and prey constructs (as described in CLONTECH YEAST protocol hand book, supra). Three independent transformants were grown overnight in synthetic media (Leu-Trp), re-suspended in Z buffer and lysed in liquid nitrogen. Subsequently, Z buffer containing the ONPG substrate and β-mercaptoethanol was added, and culture tubes were incubated at 30° C. Reactions were terminated upon addition of 1M Na₂CO₃ prior to centrifugation, and spectrophotometric analysis of supernatants at wavelength of 420 nm.

RNA extraction and Northern analysis—Total RNA was obtained from the indicated tissues using the LiCV/urea precipitation method. The RNA was separated on 1.2% agarose, and transferred to a Magna nylon membrane. The filters were hybridized with a randomly primed Ta1 probe or GAPDH. Hybridization was done according to standard procedures [Sambrook et al., (1989) Molecular Cloning: A Laboratory Manual, 2nd edn (Cold Spring Harbor, Cold Spring Harbor Laboratory Press)], and the filters were washed in 0.1×SSC and 0.1% SDS at 60° C. RNA products were revealed by autoradiography.

Immunofluorescence—Transfected cells grown on cover slips were fixed for 15 min with 3% paraformaldehyde in PBS, washed with PBS, and permeabilized for 10 min at room temperature with PBS containing 1% bovine serum albumin and 0.2% Triton X-100. For staining, cover slips were incubated for 1 hr at room temperature with anti-Flag, anti-Tsg101 or anti-HA mAbs, either alone or in combination. After extensive washing in PBS, the cover slips were incubated at room temperature for 40 min with Cy3- or Cy2-conjugated donkey anti-mouse F(ab)₂, either alone or in combination with Cy3-conjugated donkey anti-rat F(ab)₂. For EGF uptake, prior to fixation, transfected cells were washed in PBS and incubated in starvation medium (DMEM supplemented with 0.05% FCS) for 3hr, after which cells were washed once with binding buffer (DMEM supplemented with 1% bovine serum albumin and 20 mM HEPES (pH 7.5)) and incubated with binding buffer containing 2 μg/ml of EGF-conjugated to Alexa Fluor 488 (Molecular Probes) at 4° C. for 30 min. After binding, cells were transferred to 37° C. for the indicated time points, washed in PBS and fixed as described above. After staining, cover slips were mounted in moviol, and immunofluorescence was viewed and analyzed using a Confocal Zeiss Axiovert 100 TV microscope with a 63×/1.4 plane Apochromat objective, attached to the Bio-Rad Radiance 2000 laser scanning system, operated by LaserSharp software, or a Zeiss Axioplan microscope equipped with SPOTII.

In vitro ubiquitylation assays—For self-ubiquitylation, HA-tagged Ta1 proteins were immunoprecipitation from cleared extracts of transfected HEK-293T cells. Following isolation, agarose beads were extensively washed with HNTG buffer [20 mM HEPES (pH 7.5), 150 mM NaCl, 0.1% Triton X-100, and 10% glycerol], followed by an additional wash with ubiquitin wash buffer [5 mM MgCl₂ 50 mM Tris-HCl (pH 7.5), 2 mM dithiotreitol, and 2 mM ATP ). The agarose beads were then re-suspended in buffer containing 5 mM MgCl₂, 50 mM Tris-HCI pH 7.5, 2 mM dithiotreitol, 2 mM ATP and ¹²⁵I-Ubiquitin (0.5 μg per reaction). Ubiquitination assays were carried out by adding purified E1l (160 ng), and E2 (UBC-H5C; 5 μl of crude bacterial extract). Reaction mixtures were incubated for 1 hour at 30° C. The beads were then extensively washed with HNTG buffer and proteins were eluted and resolved by gel electrophoresis. For trans-ubiquitylation, Flag peptide-tagged Tsg101 was immunoprecipitated from cleared extracts of HEK-293T cells. Immunoprecipitates were extensively washed with HNTG buffer, and tumbled for 1.5 hr at 4° C. with cleared extracts of HEK-293T cells expressing HA-Ta1, to allow formation of Tsg101-Ta1 complexes. Thereafter, the beads and associated proteins were washed with ubiquitin wash buffer, and ubiquitylation assays performed as described above.

Preparation of virion-like particles (VLPs)—HEK-293T cells were transfected with 0.5 μg of vector encoding Gag-GFP in six well plates using the calcium phosphate method. Culture media were collected after 24-36 hrs. VLPs were pelleted by layering 1.2 ml of the total 2 ml of culture medium onto 200 μl of 20% sucrose (in PBS), and centrifuging at 13,000 rpm for 90 min. Alternatively, the 2 ml of cell medium was centrifuged for 5 min at 14,000 rpm, filtered through a 0.45 μm filter and VLPs were pelleted by centrifugation for 2 hr at 14,000 rpm. In both cases, the VLPs pellet was resuspended in 25 μl of 2×SDS sample buffer, boiled, resolved by SDS-PAGE and subjected to Western blot analysis. The two methods of VLP isolation yielded identical results.

Small inhibitory RNAs—The following siRNAs duplexes were synthesized: Tsg101: sense, 5′-CCUCCAGUCUUCUCUCGUCdTdT-3′(SEQ ID NO: 41) and antisense, 5′-dTdTGGAGGUCAGAAGAGAGCAG-3′(SEQ ID NO: 42), Control: sense, 5′-GUCCAAAGGWUCCGGAGACdTdT-3′(SEQ ID NO: 43) and antisense, 5′-dTdTCAGGUUUCCAAGGCCUCUG-3′(SEQ ID NO: 44). Two 21 nucleotide-long RNA duplexes corresponding to hTa1 (Genebank BC009239) coding nucleotides 272-290 and 1252-1270, relative to the starting codon were designed. Ta1 siRNA sequences are as follows: 272 sense: 5′-UCACCUCACUUCCCUGCUUdTdT-3′(SEQ ID NO: 45); 272 antisense: 5′-dTdTAGUGGAGUGAAGGGACGAA-3′(SEQ ID NO: 46); 1252 sense: 5′-UGCUGACUGAGAGCUGUAAdTdT-3′(SEQ ID NO: 47); 1252 antisense: 5′-UUACAGCUCUCAGUCAGCAdTdT-3′(SEQ ID NO: 48); control Ta1 siRNA sequences (scrambled 1252) are as follows: sense: 5′-AAUGUCGAGAGUCAGUCGUdTdT-3′(SEQ ID NO: 49) ; antisense, 5′-ACGACUGACUCUCGACAUUdTdT-3′(SEQ ID NO: 50). All the siRNAs were chemically synthesized, purified, and annealed by Dharmacon Research (Dharmacon Research, Lafayette, Colo.). Usage of the siRNAs was according to the manufacturer's instructions. Cells were transfected with the relevant double-stranded siRNA (50 nM) using Lipofectamine 2000 (Invitrogen).

Metabolic labeling of cultured cells—Transfected CHO cells were rinsed twice and pre-incubated for 3 hours in cysteine- and methionine- free medium supplemented with 10% dialyzed serum. Thereafter, cells were labeled for 15 minutes with a mixture of ³⁵S-labeled amino acids (pulse). Cells were then washed thoroughly, and incubated in media containing non-labeled cysteine and methionine for the indicated time intervals (chase). This was followed by cell lysis, immunoprecipitation, electrophoresis and autoradiography.

Construction of a nucleic acid construct capable of expressing recombinant Tal-PTAP (SEQ ID NO: 51)—Two flanking Ta1 primers were used to amplify Ta1 nucleic acids 2019-2088. The PCR product was later on cleaned and cut with BamHI and EcoRI restriction enzymes. Thereafter, the pEGFP-C1 vector (Clontech) was digested with the BamHI and EcoRI restriction enzymes, and cleaned. A ligation reaction was set between the two DNA products and later transformed into E. coli. Positive colonies were sequenced. Ta1 PTAP forward primer with EcoRI site for pEGFP-C1 was as follows 5′aagaattcagaggtcgtcacccctacgg (SEQ ID NO: 52). Ta1 PTAP reverse primer with BamHI site for pEGFP-Cl was as follows 5″aaggatccctctgcagggggagcgg (SEQ ID NO: 53).

### EXAMPLE 1

### Tsg101 Interacts with a Novel Ubiquitin E3 Ligase in the Yeast Two Hybrid System

In order to identify proteins that potentially work together with Tsg101 in budding away from the cytoplasm, essentially MVB formation and retrovirus egress, a yeast two hybrid screen was effected to characterize Tsg101 interacting proteins.

Full length Tsg101 (GenBank Accession No. NM_{—}006292) fused to the LexA binding domain was used as a bait in a yeast two-hybrid screen of a human brain cDNA library. From 6.5×10⁶ transformants screened, three interacting clones were identified, of which only one was a novel protein and two were previously identified proteins known to function in the endocytic pathway. Three independent clones of the motor protein KIF5A [Aizawa et al., (1992) J Cell Biol 119, 1287-96], and two independent clones of intersectin1, a component of the endocytic machinery that contains two EH domains and five SH3 domains [O'Bryan et al., (2001) Oncogene 20, 6300-8], were found to interact with Tsg101. Interestingly, all clones isolated included the coiled-coil region of the respective proteins. The novel Tsg101 -interacting protein was named Ta1, for Tsg101 associated ligase.

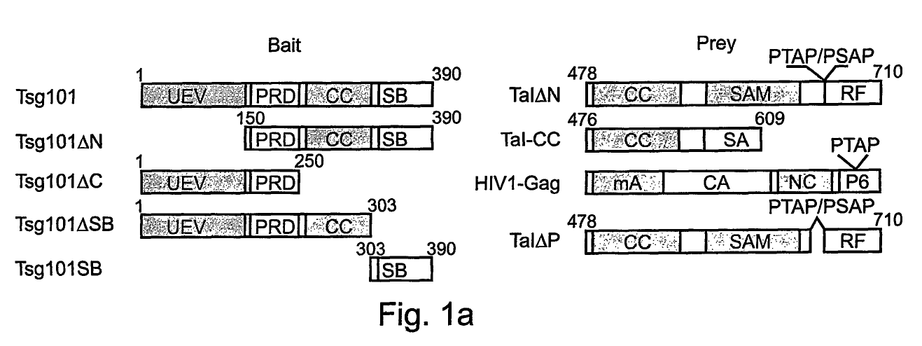

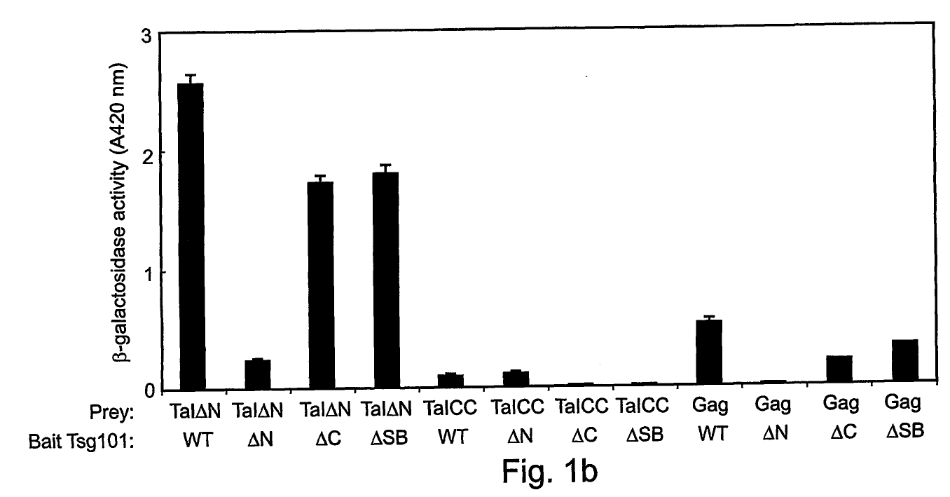

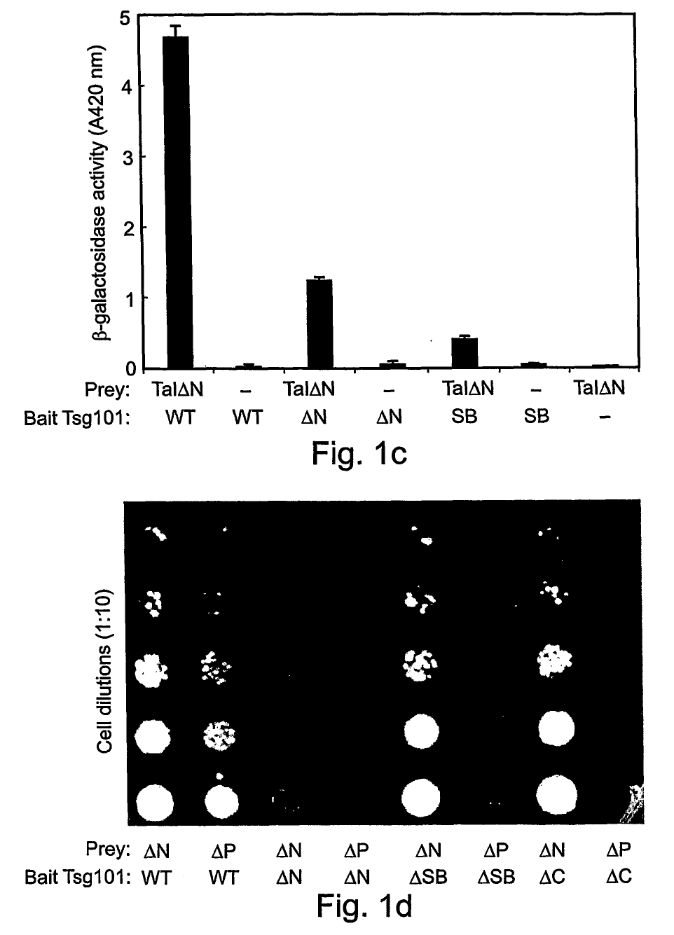

To map the interaction between Tsg101 and hTA1, various deletion mutants were constructed and their interaction was addressed in yeast (FIG. 1a). N-terminus truncated hTa1 (ΔN), which was identified in the original screen was used as a bait. HWV-Gag was used as control, since the single PTAP motif of this viral protein strongly binds to the UEV of Tsg101. Indeed, using HIV-Gag as prey, the binding of TaΔN to full-length Tsg101 was confirmed, as well as to two carboxyl terminal truncation mutants (Tsg101-ΔC and ΔSB), but not to a mutant lacking the UEV (Tsg101-ΔN; FIG. 1b). In contrast with Gag-Tsg101 interactions, despite reduced binding to hTa1, Tsg101 -ΔN reproducibly retained significant recognition. This observation indicated that hTa1 and Tsg101 maintain PTAP-dependent and PTAP-independent interactions. Secondary recognition was verified by using a mutant of hTa1 lacking the carboxyl-terminus (hTal-CC; FIG. 1b). Further analysis localized the secondary interaction to the SB of Tsg101. In line with this model, an isolated SB retained weak, but specific binding to hTa1 (FIG. 1c).

To directly address the PTAP motifs, a corresponding internal deletion mutant was examined (Ta1-ΔP; FIG. 1g). These analyses confirmed bimodal interactions, and showed that Ta1-ΔP completely lost binding to a mutant Tsg101 lacking an intact SB.

In conclusion, hTa1 and Tsg101 display bimodal interactions: in addition to the predicted UEV-PTAP recognition, the highly conserved SB mediates secondary Tal-Tsg101 interactions by binding to a central region of hTa1.

### EXAMPLE 2

### Domain Structure and Tissue Expression of Ta1 Results

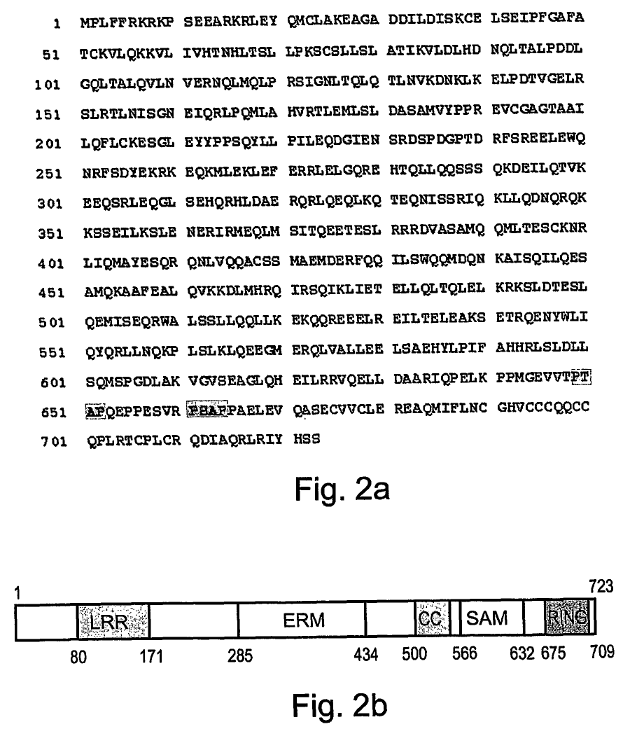

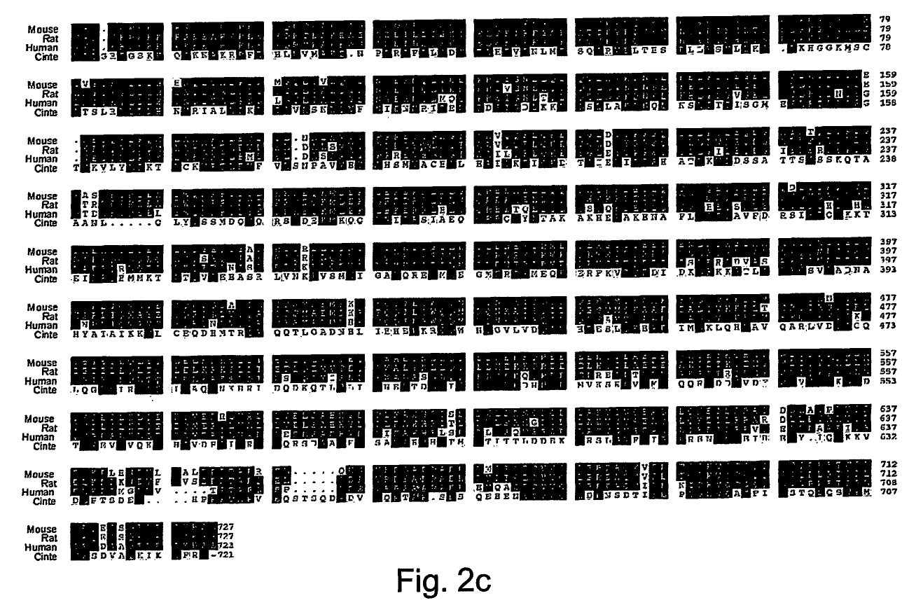

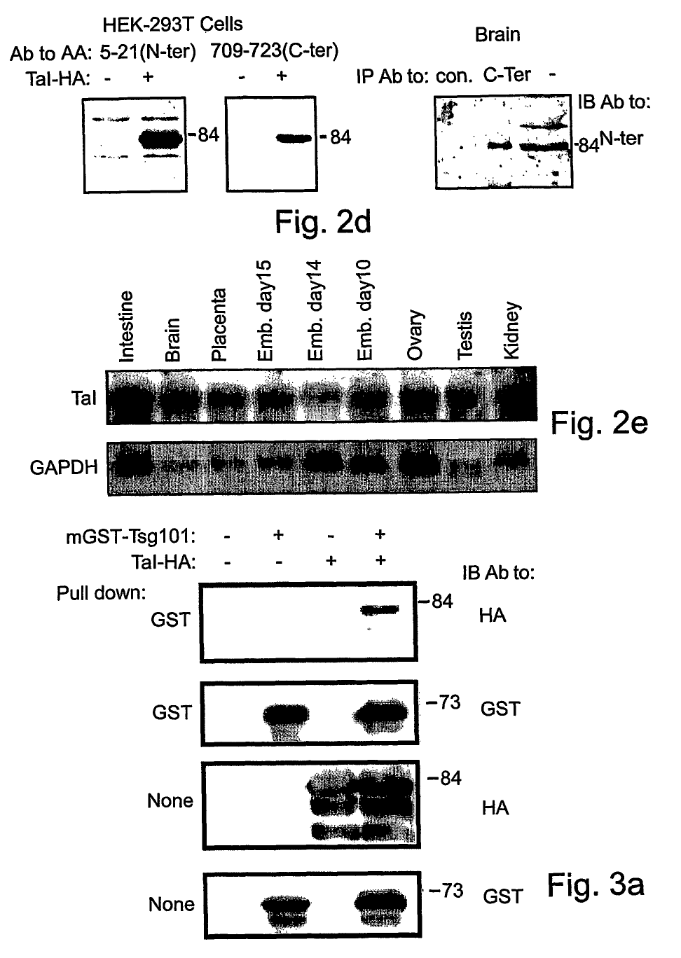

The full-length cDNA of human Ta1 was isolated from a cDNA library derived from a human breast cancer cell line, T47D. As shown in FIG. 2a, the open reading frame of human Ta1 encodes a protein of 723 amino acids. The predicted amino acid sequence of Ta1 contains an N-terminal leucine-rich repeat (LRR) followed by an ERM domain, coiled-coil region, SAM domain and a C-terminal C3HC4-type RING finger domain (FIG. 2b), which is present in many E3 ubiquitin-ligases [Joazeiro and Weissman, (2000) Cell 102, 549-52]. Interestingly, Ta1 also contains adjacent PTAP and PSAP motifs in a C-terminus portion thereof, and all four amino acids of respective motif located within the late domain of Gag proteins of multiple viruses interacts with the UEV domain of Tsg101 [Pornillos et al., (2002) Nat Struct Biol 9, 812-7; Pornillos et al., (2002) Embo J 21, 2397-406]. Furthermore, mouse Ta1 (GenBank Accession No. XM149118.3 was identified in the Ensembl data bank, and displayed 86% nucleic sequence identity, 88.6% amino acid sequence identity and 91.15% amino acid sequence homology to the human corresponding sequences as determined by recursive Blast analysis using default parameters (www.ncbi.nlm.nih.gov/homloGene, FIG. 2c). Similar level of homology was exhibited with rat Ta1 (GenBank Accession No. XM231157.1, nucleic acid identity 87.9%, amino acid sequence identity 89.48% and amino acid sequence homology 92.5%). As is evident from FIG. 2c, Ta1 is conserved only in vertebrates.

To confirm the amino acid sequence of Ta1, antibodies to synthetic peptides derived from the N- and the C-terminus of hTa1 were raised. The antibodies identified both an ectopic hTa1 transiently over-expressed in HEK-293T cells, and a similar 80 kDa species in mouse brain (FIG. 2d).

Northern blot analysis of Ta1 with RNA from various mouse tissues, shown in FIG. 2e, identified one major hybridizing band of 3.5 kb, which was detected in most tissues and developmental stages from embryonic day 10.

### EXAMPLE 3

### Ta1 and Tsg101 Interact in Mammalian Cells

In order to confirm that the interactions observed in yeast are found in mammalian cells, a series of co-immunoprecipitation experiments in HEK-293T cells transiently expressing HA-tagged Ta1 and mGST-tagged Tsg101 was effected.

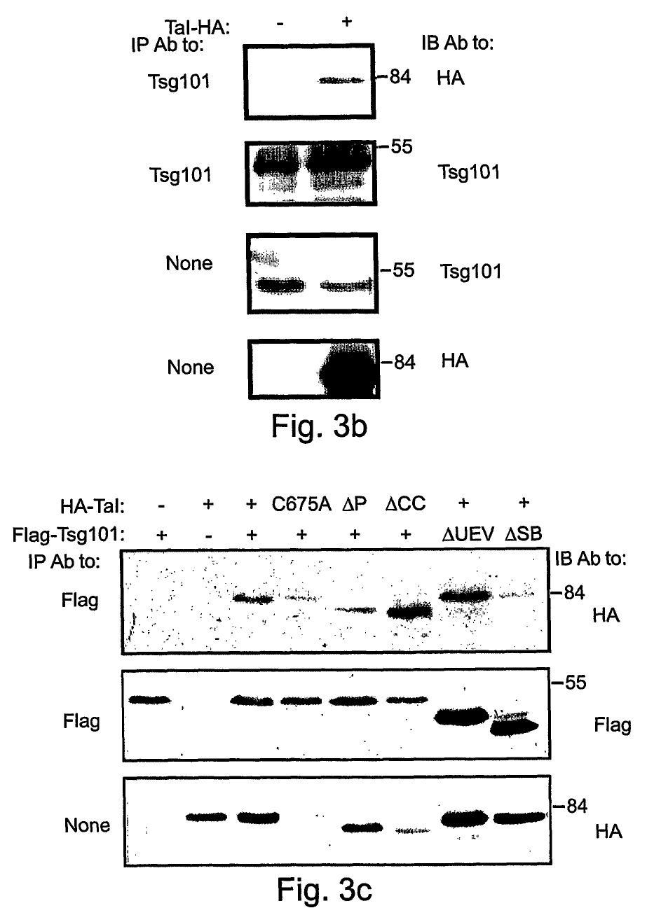

Exponentially growing transfected cells were lysed and their cleared extracts were subjected to pull-down assays with GSH-coupled agarose beads. As shown in FIG. 3a, Ta1 co-precipitated with mGST-Tsg101, while no Ta1 protein was precipitated by the beads in the absence of mGST-Tsg101. In agreement with this conclusion, the endogenous Tsg101 of HEK-293T cells underwent co-precipitation with Tsg101, which was precipitated using a specific antibody coupled to agarose (FIG. 3b). Note that endogenous Ta1 protein is consistently detected as a major band of an apparent molecular weight of 80 kDa, consistent with the molecular weight predicted by computer analysis of the mRNA sequence. Interestingly, when HA-tagged Ta1 was overexpressed, two additional HA-immunoreactive bands were observed, which may represent proteolytic cleavage.

In order to dissect the domain mediating the interaction between Tsg101 and Ta1, HEK-293T cells were co-transected with HA-Ta1 and various deletion mutants of a Flag peptide-tagged Tsg101. As shown in FIG. 3c, Ta1 co-precipitated with deletion mutants lacking the UEV, coiled-coil and proline-rich region. However, Tsg101 did not recognize a Ta1 mutant missing the SB region, confirming the data from the yeast two hybrid analyses (FIG. 1b).

### EXAMPLE 4

### Ta1 is an E3 Ubiquitin-Ligase that Ubiquitylates Tsg101 in Living Cells and in vitro

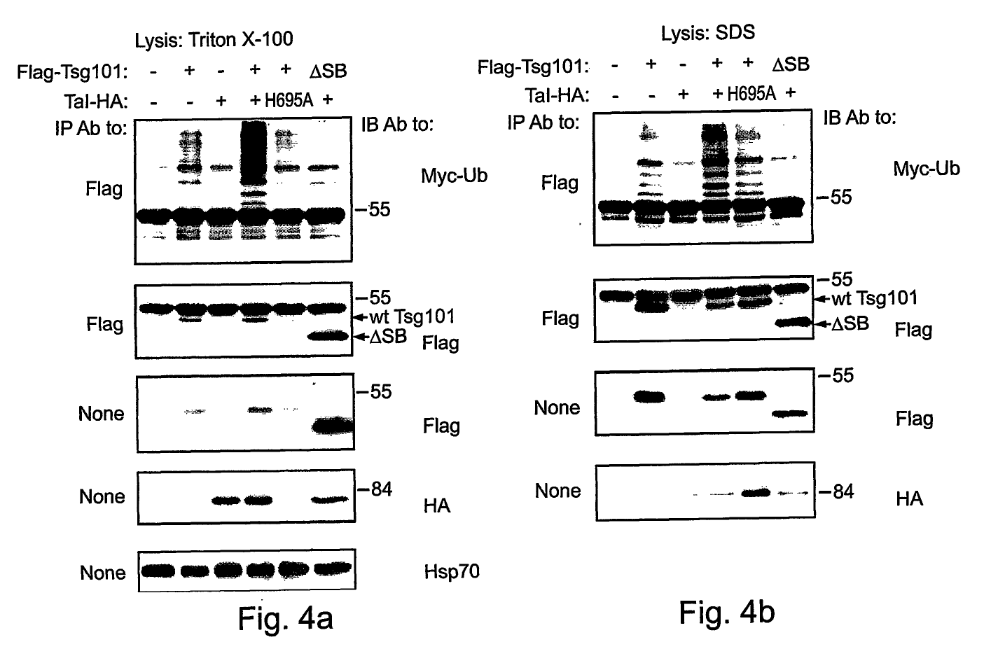

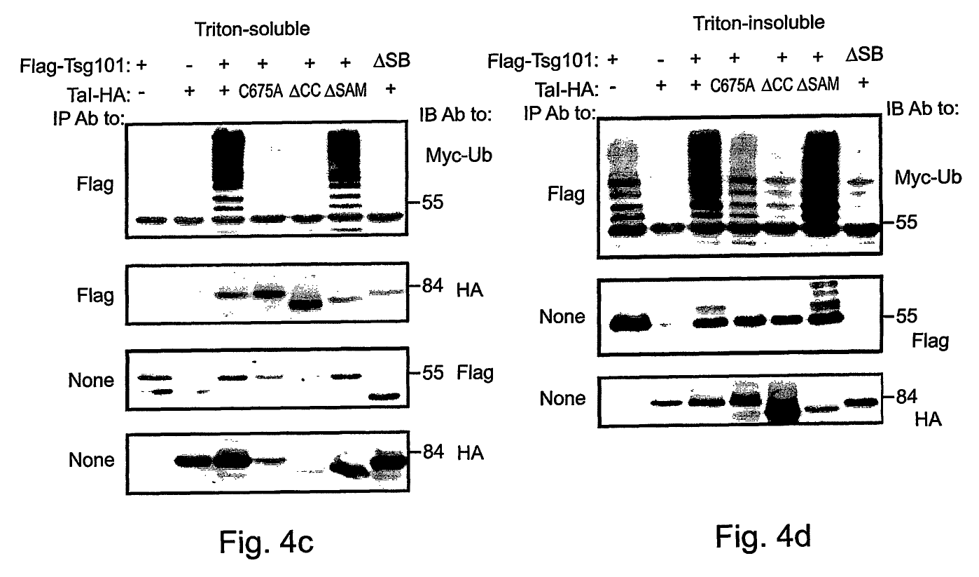

As Ta1 contains a C3HC4type RING finger domain with homology to similar motifs found within known E3 ubiquitin ligases [Joazeiro and Weissman (2000) Science 286, 309-312], the ability of Ta1 to promote ubiquitylation of Tsg101 upon interaction therewith was addressed. Ubiquitylation of Tsg101 was first addressed in HEK-293T cells co-expressing Flag-Tsg101 and Myc-tagged ubiquitin (Myc-Ub). Ubiquitylation of exogenous Tsg101 was identified by immunoblotting with anti-Myc antibodies. As shown in FIG. 4a, a high molecular weight smear representing ubiquitylated Tsg101 was apparent in cells expressing Tsg101 and Myc-Ub. Expression of an ectopic Ta1 resulted in a dramatic increase in the levels of Tsg101 ubiquitylation.

To test whether Ta1 is the E3 ligase responsible for the ubiquitylation of Tsg101, a histidine residue within the RING finger of Ta1, which is predicted to be crucial for its E3 ligase activity was mutated (mutant denoted H695A-Tal). As expected, in contrast to wild type Ta1, H695A-Ta1 did not increase Tsg101 ubiquitylation. Further, mutation of the first cysteine residue within the RING finger domain (residue 675), which is also expected to be critical for E3 ligase activity, yielded similar results. As the steadiness box is the region of Tsg101 which mediates the association with Ta1 (FIGS. 1a-d and 3a-c), the effect of deletion of this region on Tsg101 ubiquitylation was determined.

These results suggest that Ta1 is an E3 ligase, which mediates Tsg101 ubiquitylation in a RING-finger dependent manner.

Notably, an apparent low expression of both H695A-Ta1 and Tsg101 (wild type) in cells expressing this Ta1 mutant (FIG. 4a; lower two panels), and immunoblotting with a control antibody (to heat shock protein 70) verified equal gel loading. To examine the possibility that the observed differences were due to translocation of Tsg101 to an insoluble fraction Triton X-100 was replaced with the ionic detergent sodium dodecylsulfate (SDS). This analysis revealed that H695A-Tal lost detergent solubility, rather than expression levels (see anti-HA blotted panels in FIGS. 4a and 4b). Strikingly, the majority of Tsg101 was found to be insoluble in Triton X-100 FIG. 4b, and data not shown), but ubiquitylation thereof significantly increased solubility. For example, mono- and oligo-ubiquitylated forms of Tsg101 were less soluble than poly-ubiquitylated forms in Triton X-100, but SDS-exhibited solubilities were comparable. A mutant lacking the SB was mis-localized and it displayed high solubility, but no ubiquitylation (FIG. 4a).

These observations imply that along with enabling ubiquitylation, the SB links Tsg101 to insoluble structures. Hence, hTa1 controls both ubiquitylation and solubility of Tsg101. Indeed, experiments in which cells were first extracted with Triton X-100, and the insoluble fraction was then treated with SDS, verified that the solubility of Tsg101 correlates with its ubiquitylation by hTa1 (FIGS. 4c-d): Tsg101 is less soluble in the presence of the ubiquitylation-defective, and relatively insoluble hTa1-C675A. Further, deletion of the coiled-coil (ΔCC), but not the SAM region of hTa1, severely reduced ubiquitylation of Tsg101 and solubility of both Tsg101 and hTa1. Last, in line with bimodal Tsg101-Ta1 interactions, deletion of the PTAP motifs of hTa1 abolished binding to and ubiquitylation of Tsg101 (FIG. 5a). This mutant was as soluble as WT hTa1 in Triton X-100, and it did not affect solubility of Tsg101.

To ascertain self-ubiquitylation of hTa1, an activity shared by many RING-bearing ligases, hTa1 mutants were co-expressed together with a Flag-tagged ubiquitin. As expected, WT-hTa1, as well as mutants lacking the CC and PTAP motifs exhibited a smear of ubiquitylated species, but a RING-defective mutant was not modified (FIG. 5b). In vitro auto-ubiquitylation of hTa1 was tested by incubating the isolated protein in the presence of recombinant E1, E2, and ¹²⁵I-labeled ubiquitin. This analysis confirmed that hTa1 is capable of self-ubiquitylation, which is dependent on an intact RING (FIG. 5c).

A similar protocol was used to assess the ligase activity of hTa1 towards Tsg101. An immunoprecipitated Tsg101 was pre-incubated with extracts derived from cells overexpressing wild type or mutant hTa1 proteins, and the Tsg101-Ta1 complexes were subjected to an in vitro ubiquitylation assay. Substantial Tsg101 ubiquitylation was observed in complexes pre-incubated with WT-hTa1, whereas only background ubiquitylation was observed with two RING mutants of hTa1 (FIG. 5d). Taken together with experiments performed in living cells, these results identity hTa1 as a physiological E3 ubiquitin ligase for Tsg101.

Unlike proteasomal degradation, which requires a chain of ubiquitins polymerized through lysine 48 of ubiquitin, conjugation of monomeric ubiquitins is associated with vesicular sorting [Hicke, (2001) Nat Rev Mol Cell Biol 2, 195-2]. To determine whether the smeary appearance of ubiquitylated Tsg101 corresponds to poly-ubiquitylation or to multiple monomeric ubiquitins (multi-ubiquitylation), a polymerization-defective mutant of ubiquitin (Ub4KR), which lacks all four branching lysines was used. Because similar patterns of ubiquitylated Tsg101 were obtained with both WT and mutant ubiquitin (FIG. 5e), a conjugation of monomeric, rather than polymeric ubiquitin was inferred. To verify conjugation of more than one mono-ubiquitin per Tsg101 molecule, Ub-4KR was fused to two different peptide tags, and co-expressed the two species in cells over-expressing Tsg101. In support of multi-ubiquitylation, a double-step isolation protocol yielded Tsg101 molecules that were simultaneously modified by two distinct mono-ubiquitins. As expected, a catalytically-defective mutant of hTa1 (C675A) eliminated the ubiquitylation signal in these experiments, primarily by reducing protein solubility.

In conclusion, hTa1 mediates mono-ubiquitylation of Tsg101, consistent with a role in vesicular trafficking.

### EXAMPLE 5

### Ta1 partly Co-Localizes with Tsg101 to a Sub-Membranal Domain

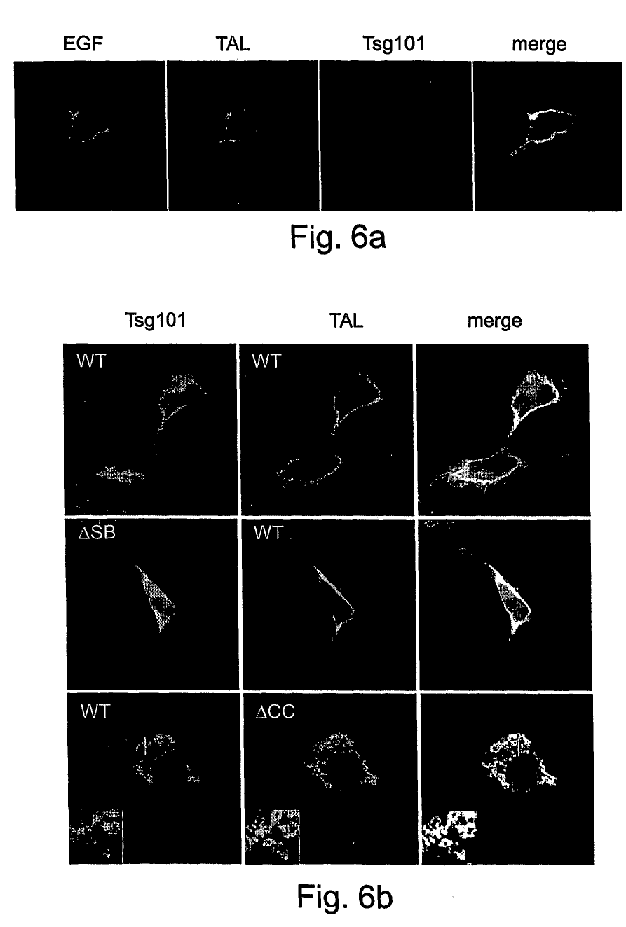

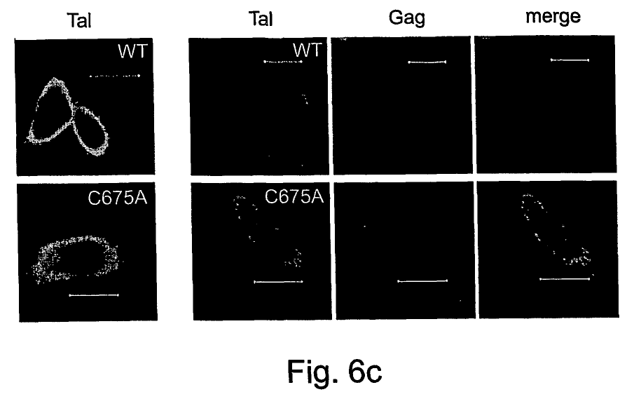

The subcellular localization of Ta1 and Tsg101 was addressed using confocal microscopy (FIGS. 6a-c). As previously reported [Bishop and Woodman, (2001) J Biol Chem 276, 11735-42], Tsg101 showed a vesicular localization corresponding to endosomal clusters (FIG. 6a). However, these structures were devoid of Ta1. Binding of fluorescent EGF to the outer plasma membrane under conditions that prevent EGFR internalization revealed that hTa1 localizes to the inner leaflet of the plasma membrane. When expressed at moderate levels, Tsg101 displayed sub-membranal, reticular and endosomal punctate localizations FIG. 6b), in line with previous reports [Bishop and Woodman, (2001) supra; Goila-Gaur et al., (2003) J Virol 77, 6507-6519]. The sub-membranal fraction of Tsg101 partly co-localized with hTa1. The endosomal localization of Tsg101 was abolished upon deletion of the SB, which may underlie its complete solubility in Triton X-100 (FIGS. 4a-d), and the corresponding mutant did not affect hTa1's distribution. In contrast, hTa1 mutants defective in the RING or CC domains mis-localized, together with WT-Tsg101, to the outer rim of large vesicular structures (FIG. 6b, insets; and data not shown).

### EXAMPLE 6

### Ta1 Synergizes with Tsg101 to Inhibit Release of HIV-1 Gag

Because Tsg101 is crucial for budding of HIV-1 and Ebola viruses from the cell membrane [Demirov et al., (2002) Proc Natl Acad Sci USA 99, 955-60; Garrus et al., (2001) Cell 107, 55-65; Martin-Serrano et al., (2001) Nat Med 7, 1313-9], and due to the profound effects of Ta1 on Tsg101 ubiquitylation and localization, it was postulated that Ta1 might also affect the function of Tsg101 with respect to viral budding.

To test this possibility, the two most commonly used cellular systems for HIV1 gag budding, HEK-293T and HeLa cells, were used.

HEK-293T cells were transfected with GFP-Gag fusion protein. The Gag poly-protein is the major structural protein of retroviruses and is entirely sufficient for particle formation′[reviewed in Swanstrom and Erona, (2000) Pharmacol Ther 86, 145-70]. Hence, GFP-Gag mimics normal Gag budding. As shown in FIGS. 7a-c, overexpression of either Ta1 or Tsg101 reduced GFP-Gag secretion into virus-like particles (VLPs) by 50%, whereas overexpression of both reduced viral budding by over 90% (FIG. 7a; at both 24 hrs and 36 hours). The effects of Ta1 on viral budding were mediated through Tsg101 ubiquitylation, as no effect was observed with either catalytically inactive Ta1 (H695A) or upon expression of ΔSB-Tsg101, which could not interact with Ta1. Tsg101 was reported to be included within virus-like particles [Myers and Allen, (2002) J Virol 76, 11226-35]. Interestingly, in the presence of Tsg101, H695A-Ta1 but not wild type Ta1 was also seen in VLPs (right second panel from top).

Interestingly, unlike WT-hTa1, all ubiquitylation-defective mutants of hTa1, namely H695A, C675A and ΔCC, were detectable in VLPs (FIGS. 7a and 7b). Presumably, a Tsg101 -hTa1 complex escorts Gag into viral particles, but the complex is dissociated, and Ta1 escapes exocytosis upon ubiquitylation of Tsg101 or Gag.

In order to better understand the counter effects of HIV-1 Gag on Ta1 and vis versa, GFP-Gag was expressed in HeLa-SS6 cells. When co-expressed with WT-hTa1, Gag significantly narrowed the sub-membranal distribution of hTa1 to a fine peripheral layer containing both proteins (FIG. 7e). In contrast to WT-hTa1, the mis-localized C675A-hTa1 altered the distribution of Gag, and both co-localized to circular structures similar to those observed with other dominant negative mutants of Ta1.

Gag contains a PTAP motif, which recruits Tsg101 to virus budding sites [Garrus et al. (2001) Cell 107:55-65; Martin-Serrano et al. (2001) Nat. Med. 7:1313-9; VerPlank et al. (2001) Proc. Natl. Acad. Sci. USA 98:7724-9; Demirov et al. (2002) Proc. Natl. Acad. Sci. USA 99:955-60; Myers and Allen (2002) J. Virol. 76:11226-35; Pornillos et al. (2002a) Nat Struct Biol. 9:812-7]. In addition, it has been shown that Tsg101 multimerizes via its CC domain (Martin-Serrano et al. 2003, supra). Hence, the observed co-distribution of hTa1 and GFP-Gag might be mediated by a Gag-(Tsg101)₂-Ta1 complex. Indeed, the Gag polyprotein (Pr55^{Gag}) and Ta1 underwent co-immunoprecipitation when co-expressed, but blocking expression of Tsg101 by using a specific siRNA (SEQ ID NOs: 41-44) practically abolished complex formation (FIG. 7f). Hence, it is likely that a Tsg101-Ta1 complex is recruited by Gag to sites of virus egress.

This conclusion predicts insertion of Ta1 into VLPs, as has been reported for Tsg101 [Myers and Allen (2002) J. Virol.76:11226-35].

Indeed co-budding experiments performed in HEK-293T cells supported budding of hTa1. Unlike a PTAP deletion mutant of hTa1, which cannot bind Tsg101, WT-hTa1 was detectable in VLPs (FIG. 7g). Moreover, more effective insertion into VLPs was observed with C675A-hTa1 (FIG. 7g), a ubiquitylation-defective mutant that strongly binds to and decreases solubility of Tsg101 (FIG. 4c).

These observations suggested that a stable, perhaps un-ubiquitylated, Tal-Tsg101 complex is essential for insertion of Ta1 into VLPs. Hence ΔCC-hTa1, an ubiquitylation-defective mutant that binds Tsg101 even stronger than C675A-hTa1 was tested. Extremely effective insertion of ΔCC-hTa1 into VLPs was observed (FIG. 7b), which reinforces the interpretation that Tsg101 drives Ta1 into budding HIV-1 particles.

Because Tsg101 plays an essential role in budding of HIV-1 (Garrus et al. 2001; Martin-Serrano et al. 2001; Demirov et al. 2002 supra) as Ta1 profoundly alters Tsg101 partitioning by means of multi-ubiquitylation (FIGS. 4a-d and 5a-e), Ta1's effect on VLP release was addressed. A Ta1-specific siRNA (denoted Ta1-1252) dramatically increased budding (FIG. 7h), consistent with ubiquitylation-mediated inactivation of Tsg101. Interestingly, only limited inhibition of VLP release was observed in cells overexpressing hTa1, but co-expression of Ta1 and Tsg101 almost blocked egress (FIG. 7a).

Ubiquitin plays an important, albeit poorly understood role in viral budding (Ott et al., (2003), J Virol 77, 338493; Schubert et al., (2000) Proc Natl Acad Sci USA 97, 13057-62]. Hence, a role for hTa1 and Tsg101 in Gag ubiquitylation was then determined. As shown in FIG. 7c, overexpression of Tsg101 significantly increased ubiquitylation of p55-Gag. Similarly, it has been reported that overexpression of Tsg101 increases ubiquitylation of HIV-2 Gag [Myers and Allen, (2002), J Virol 76, 11226-35]. Co-expression of hTa1 with Tsg101 decreased Gag ubiquitylation to the basal level, presumably because the stoichiometry of a transient Ta1-Tsg101 -Gag complex was disrupted. Consistent with this possibility, catalytically-defective forms of hTa1 (i.e., ΔCC and C675A) acted as dominant negative mutants that reduce Gag ubiquitylation below the background level (FIG. 7c, and data not shown). Further, when co-expressed with these mutants, the Gag protein lost solubility. In conclusion, because inactive mutants of hTa1 reduce Gag ubiquitylation and they are co-exocytosed with Gag, it is conceivable that transient Gag-Tsg101 -Ta1 complexes are necessary for budding, whereas blocking complex dissociation, or interrupting its stoichiometry, inhibits Gag ubiquitylation, and consequently reduces budding efficiency.

In many assembly-defective Gag mutants, processing of the Gag precursor is impaired. Notably, the ΔCC mutant of hTa1 impaired processing of p55-Gag to the p24 species (FIG. 7c), suggesting that in cells expressing this hTa1 mutant, Gag molecules may assemble incorrectly. To test this prediction, a single-cycle infection assay in cells expressing a mixture of three plasmids that generate an infectious, GFP-encoding vector based on HIV-1 was employed [Naldini et al., (1996) Proc Natl Acad Sci USA 93, 11382-11388]. VLP-containing supernatants were used to infect naive cells, whose GFP fluorescence was determined. The results presented in FIG. 7d indicate that overexpression of hTa1 reduces infectivity by approximately 40%, but when combined with an ectopic Tsg101, hTa1 exerted a significantly greater inhibitory effect. RING or CC mutants of hTa1 potently inhibited HIV-1 infectivity even in the absence of an ectopic Tsg101, probably reflecting interactions with the endogenous Tsg101 protein. Interestingly, a PTAP-defective mutant of hTa1, which only weakly binds and ubiquitylates Tsg101, mediated a relatively mild effect on viral infectivity. Conceivably, through complex formation with and ubiquitylation of Tsg101, hTa1 plays a critical role in Gag polyprotein assembly and egress to generate a fully infectious HIV.

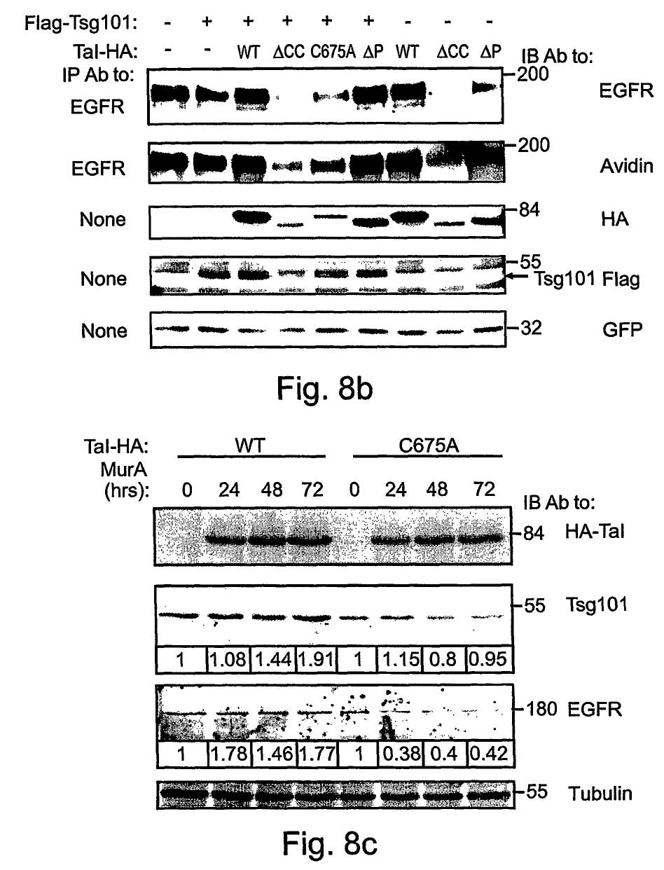

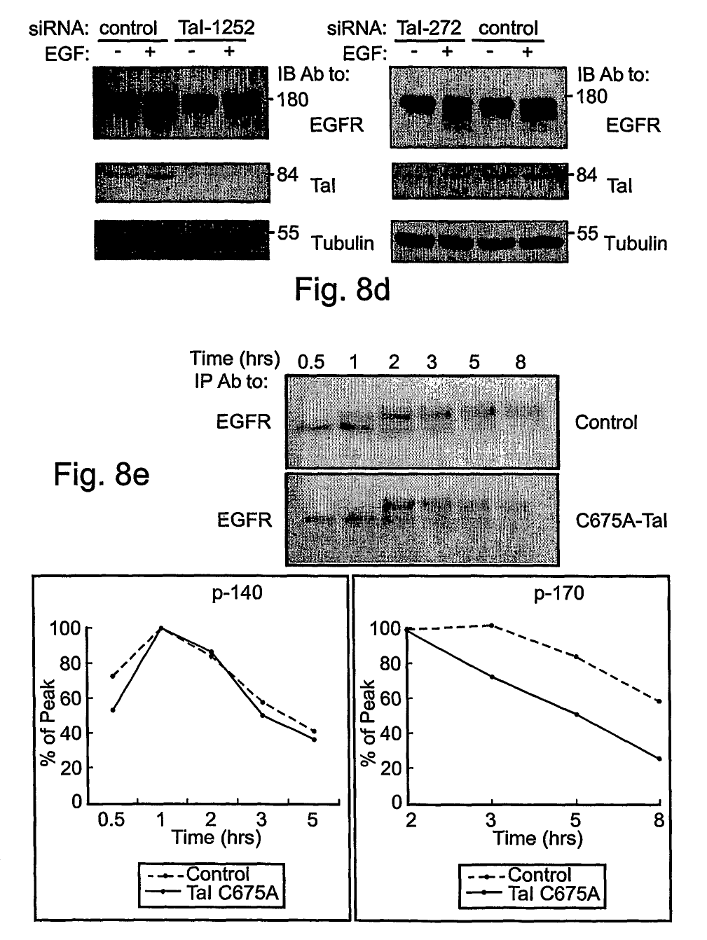

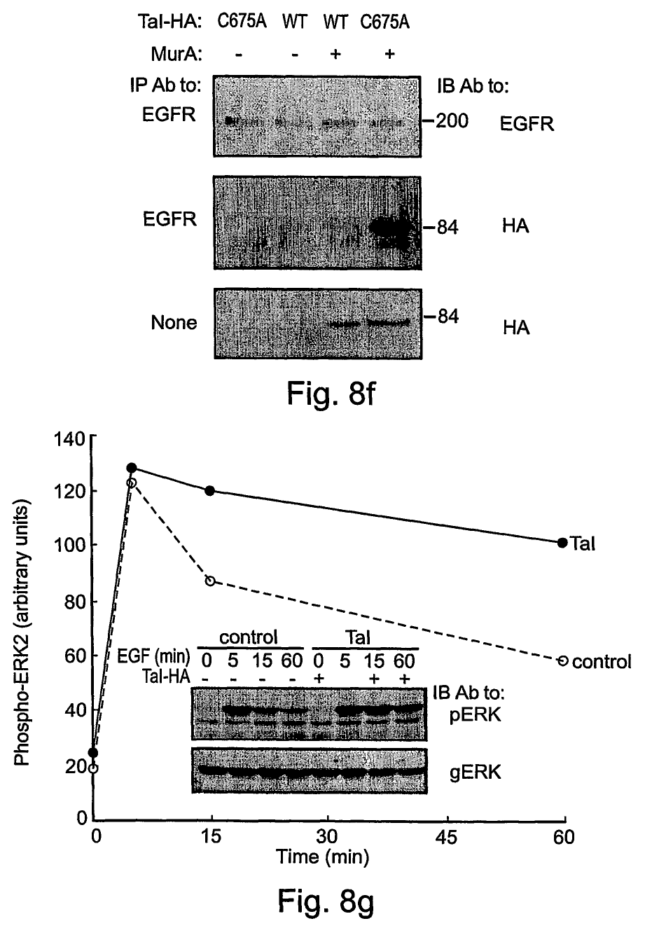

In conclusion, Ta1-mediated ubiquitylation of Tsg101 likely inactivates its virus release function, in analogy to the inhibitory action of Ta1 on Tsg101 function in late-stage endocytosis (FIGS. 8a-e further described below).

### EXAMPLE 7

### Ta1-Mediated Unbiquitylation of Tsg101 Controls Sorting Event in EGFR Endocytosis

Normal internalization of EGFRs was observed in fibroblasts depleted of Tsg101, but instead of trafficking to lysosomes, EGFRs were shunted in the MVB to a recycling pathway [Babst et al., (2000) Traffic 1, 248-58]. To analyze possible roles for hTa1 in endocytosis, a fluorescent derivative of EGF was used.

Notably, overexpression of hTa1 did not affect the rapid translocation of membranal EGFRs to endocytic vesicles (FIG. 8a, and data not shown). While the majority of hTa1 remained close to the plasma membrane, a fraction co-localized with the internalized EGFR In contrast, no co-localization was observed in cells expressing a catalytically-defective mutant of hTa1 (H695A). Surprisingly, in some experiments a reduced binding of the fluorescent derivative of EGF to cells expressing catalytically-inactive mutants of hTa1 was observed (data not shown).

Consistent with this observation, two dominant negative mutants of hTa1 (C675A and ΔCC) accelerated degradation of surface biotinylated EGFRs, even in the absence of EGF (FIG. 8b). To rigorously link the endocytic fate of EGFR to Ta1, a HEK-293T cell system that expresses a catalytically inactive mutant of hTa1 from the inducible ecdysone promoter was established. As shown in FIG. 8c, upon induction with Muristerone A, C675A-hTa1 reduced expression of the endogenous EGFR of HEK-293T cells. Consistently, WT-hTa1 moderately increased receptor stability and enhanced Tsg101 levels by increasing protein solubility. These results were further substantiated by a Tal-specific small interfering RNA sequences (siRNA). Two siRNA oligonucleotides (SEQ ID NOs: 45, 46, 47 and 48) that effectively reduced expression of the endogenous hTa1 in human cells, as revealed by using anti-Ta1 antibodies (FIG. 8d) were identified. When transfected into HeLa-SS6 cells, these oligonucleotides significantly accelerated EGF-induced degradation of endogenous EGFR molecules, consistent with a role for hTa1 in restraining late sorting of EGFR to degradation.

To test the prediction that hTa1 regulates endocytic degradation of EGFR, rather than maturation and delivery to the plasma membrane, EGFR was subjected. to a short pulse of metabolic labeling and subsequent receptor maturation and degradation were followed (FIG. 8e). As shown in FIG. 8e, exogenously expressed C675A-hTa1 did not affect the rate of synthesis of the precursor p140^{EGFR}, which gradually matured to p170^{EGFR}. The latter, however, disappeared more rapidly in the presence of C675A-hTa1.

In conclusion, these results attribute to hTa1 a role in late endocytic sorting of internalized EGFRs, and together with observations made with HIV-Gag, they support a general function in budding away from the cytoplasm.

The mechanism underlying the ability of catalytically inactive mutants of hTa1 to accelerate endocytic degradation of EGFR was then addressed. According to one model, Tsg101 directly binds mono-ubiquitylated cargoes like EGFR, and sorts them to the lumen of MVBs [Katzmann et al. (2002) Cell 106:145-55]. Presumably, the sorting function of Tsg101 is inactivated upon ubiquitylation by Ta1, but catalytically defective forms of Ta1 can maintain Tsg101 in its active, de-ubiquitylated state. This model predicts formation of a stable sorting complex containing both EGFR and C675A-hTa1.

Indeed, by using the above-described inducible hTa1, a co-immunoprecipitate including EGFR with C675A-hTa1 was detected (FIG. 8f). No complex was detectable in cells expressing WT-hTa1 or in un-induced cells, in line with the proposed model of Ta1's action.

Since WT-hTa1 is able to stabilize EGFR at the cell surface (FIGS. 8d and 8e), the effect of Ta1 on EGFR-mediated signaling, a process regulated primarily through receptor endocytosis was addressed [reviewed in Waterman and Yarden (2001) FEBS Lett. 490:142-52]. Indeed, the onset phase of MAPK activation by EGF was similar in control and in hTal-expressing cells, but the inactivation phase almost disappeared when hTa1 was over-expressed (FIG. 8g). Notably, a similar behavior of MAPK signaling was observed in cells defective in Tsg101 [Babst et al. (2000) Traffic 1:248-58], in line with the proposition that Ta1 inactivates Tsg101 by means of multi-ubiquitylation.

### EXAMPLE 8

### Inhibition of HIV-1 Budding using a Ta1-Derived Peptide

Experimental Procedures and Results

As budding of the HIV-1 Gag is dependent on active Tsg101 complexes, it was hypothesized that sequestering Tsg101 from HIV-1 Gag will inhibit budding. Since Tsg101 binds more effectively to a double PTAP motif (VerPlank et al 2001, supra), the two adjacent PTAP-PSAP motifs of Ta1 were included in a GFP-fusion peptide (SEQ ID NO: 51).

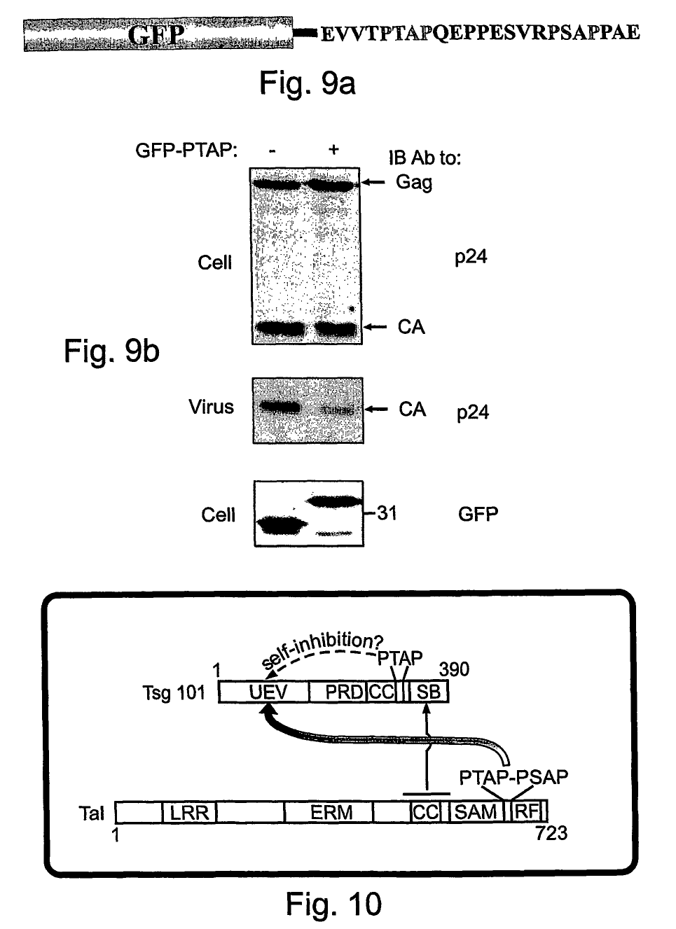

Using flanking primers, the region encompassing nucleotides 2019-2088 of ta1 was amplified and ligated into the pEGFP-C1 vector (Clontech, FIG. 9a). As shown in FIG. 9b, co-expression of GFP-PTAP along with the HIV-1 Gag completely blocked budding as compared to control expressing HIV-1 Gag along with a GFP empty vector. These results substantiate the PTAP-PSAP peptide (SEQ ID NO: 51), as a valuable anti HIV therapeutic tool.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. An isolated polynucleotide encoding a polypeptide having a sequence of at least 10 and no more than 500 amino acids, wherein said sequence is derived from the amino acid sequences of SEQ ID NO: 2, 4 or 6.

2. The isolated polynucleotide of claim 1, wherein said sequence is as set forth in amino acid coordinates 490-723 of SEQ ID NO: 2.

3. The isolated polynucleotide of claim 1, wherein said sequence is as set forth in amino acid coordinates 647-723 of SEQ ID NO: 2.

4. The isolated polynucleotide of claim 1, wherein said sequence is as set forth in amino acid coordinates 647-665 of SEQ ID NO: 2.

5. The isolated polynucleotide of claim 1, wherein said sequence is as set forth in amino acid coordinates 647-667 of SEQ ID NO: 2.

6. The isolated polynucleotide of claim 2, wherein said sequence said polypeptide is encoded by nucleotide coordinates 1556-2255 of SEQ ID NO: 1.

7. The isolated polynucleotide of claim 3, wherein said sequence of said polypeptide is encoded by nucleotide coordinates 2025-2255 of SEQ ID NO: 1.

8. The isolated polynucleotide of claim 4, wherein said sequence of said polypeptide is encoded by nucleotide coordinates 2025-2079 of SEQ ID NO: 1.

9. The isolated polynucleotide of claim 5, wherein said sequence of said polypeptide is encoded by nucleotide coordinates 2019-2088 of SEQ ID NO: 1.

10. A nucleic acid construct comprising the isolated polynucleotide of claim 1.

11. The nucleic acid construct of claim 10, further comprising a promoter for regulating transcription of the isolated polynucleotide in sense or antisense orientation.

12. The nucleic acid construct of claim 10, further comprising a positive and a negative selection markers for selecting for homologous recombination events.

13. A host cell comprising the nucleic acid construct of claim 10.

14. An isolated polynucleotide as set forth in SEQ ID NO: 38, 39 or 40.

15. An isolated polypeptide comprising an amino acid sequence of at least 10 and no more than 500 amino acids, wherein said amino acid sequence is derived from SEQ ID NO: 2, 4 or 6.

16. The isolated polypeptide of claim 15, wherein said amino acid sequence is as set forth in SEQ ID NO: 7, 8, 37 or 51.

17. The isolated polypeptide of claim 15, wherein said amino acid sequence is as set forth in amino acid coordinates 490-723 of SEQ ID NO: 2.

18. The isolated polypeptide of claim 15, wherein said amino acid sequence is as set forth in amino acid coordinates 647-723 of SEQ ID NO: 2.

19. The isolated polypeptide of claim 15, wherein said amino acid sequence is as set forth in amino acid coordinates 647-665 of SEQ ID NO: 2.

20. The isolated polypeptide of claim 15, wherein said amino acid sequence is as set forth in amino acid coordinates 647-667 of SEQ ID NO: 2.

21. The isolated polypeptide of claim 17, wherein said amino acid sequence is encoded by nucleotide coordinates 1556-2255 of SEQ ID NO: 1.

22. The isolated polypeptide of claim 18, wherein said amino acid sequence is encoded by nucleotide coordinates 2025-2255 of SEQ ID NO: 1.

23. The isolated polypeptide of claim 19, wherein said amino acid sequence is encoded by nucleotide coordinates 2025-2079 of SEQ ID NO: 1.

24. The isolated polypeptide of claim 20, wherein said amino acid sequence is encoded by nucleotide coordinates 2019-2088 of SEQ ID NO: 1.

25. An antibody or an antibody fragment being capable of specifically binding a polypeptide at least 90% homologous to SEQ ID NO: 2, as determined using the BestFit software of the Wisconsin sequence analysis package, utilizing the Smith and Waterman algorithm, where the gap creation equals 8 and gap extension penalty equals 2.

26. The antibody or antibody fragment of claim 25, wherein said polypeptide is as set forth in SEQ ID NO: 2, 4 or 6.

27. A display library comprising a plurality of display vehicles each displaying at least 6 consecutive amino acids derived from a polypeptide at least 90% homologous to SEQ ID NOs: 2 as determined using the BestFit software of the Wisconsin sequence analysis package, utilizing the Smith and Waterman algorithm, where gap creation penalty equals 8 and gap extension penalty equals 2.

28. An oligonucleotide specifically hybridizable with a nucleic acid sequence as set forth in SEQ ID NO: 1.

29. The oligonucleotide of claim 28, wherein said oligonucleotide is a single or double stranded.

30. The oligonucleotide of claim 28, wherein said oligonucleotide is at least 10 bases long.

31. The oligonucleotide of claim 28, wherein said oligonucleotide is hybridizable in either sense or antisense orientation.

32. A pharmaceutical composition comprising a therapeutically effective amount of at least an active portion of a polypeptide being at least 90% homologous to SEQ ID NO: 2, as determined using the BestFit software of the Wisconsin sequence analysis package, utilizing the Smith and Waterman algorithm, where the gap creation equals 8 and gap extension penalty equals 2 or an active portion thereof and a pharmaceutically acceptable carrier or diluent.

33. The pharmaceutical composition of claim 32, wherein said polypeptide is as set forth in SEQ ID NO: 2, 4 or 6.

34. The pharmaceutical composition of claim 32, wherein said active portion of the polypeptide is as set forth in amino acid coordinates 490-723 of SEQ ID NO: 2.

35. The pharmaceutical composition of claim 32, wherein said active portion of the polypeptide is as set forth in amino acid coordinates 647-723 of SEQ ID NO: 2.

36. The pharmaceutical composition of claim 32, wherein said active portion of the polypeptide is as set forth in amino acid coordinates 647-665 of SEQ ID NO: 2.

37. The pharmaceutical composition of claim 32, wherein said active portion of the polypeptide is as set forth in amino acid coordinates 647-667 of SEQ ID NO: 2.

38. The pharmaceutical composition of claim 34, wherein said active portion of the polypeptide is encoded by nucleotide coordinates 1556-2255 of SEQ ID NO: 1.

39. The pharmaceutical composition of claim 35, wherein said active portion of the polypeptide is encoded by nucleotide coordinates 2025-2255 of SEQ ID NO: 1.

40. The pharmaceutical composition of claim 36, wherein said active portion of the polypeptide is encoded by nucleotide coordinates 2025-2079 of SEQ ID NO: 1.

41. The pharmaceutical composition of claim 37, wherein said active portion of the polypeptide is encoded by nucleotide coordinates 2019-2088 of SEQ ID NO: 1.

42. A method of treating HIV infection in a subject, the method comprising providing to a subject in need thereof a therapeutically effective amount of at least an active portion of a polypeptide being at least 90% homologous to SEQ ID NO: 2, as determined using the BestFit software of the Wisconsin sequence analysis package, utilizing the Smith and Waterman algorithm, where the gap creation equals 8 and gap extension penalty equals 2 or an active portion thereof, to thereby treat the HIV infection in the subject.

43. The method of claim 42, wherein said polypeptide is as set forth in SEQ ID NO: 2, 4 or 6.

44. The method of claim 42, wherein said active portion of said polypeptide is as set forth in amino acid coordinates 490-723 of SEQ ID NO: 2.

45. The method of claim 42, wherein said active portion of said polypeptide is as set forth in amino acid coordinates 647-723 of SEQ ID NO: 2.

46. The method of claim 42, wherein said active portion of said polypeptide is as set forth in amino acid coordinates 647-665 of SEQ ID NO: 2.

47. The method of claim 42, wherein said active portion of said polypeptide is as set forth in amino acid coordinates 647-667 of SEQ ID NO: 2.

48. The method of claim 44, wherein said active portion of said polypeptide is encoded by nucleotide coordinates 1556-2255 of SEQ ID NO: 1.

49. The method of claim 45, wherein said active portion of said polypeptide is encoded by nucleotide coordinates 2025-2255 of SEQ ID NO: 1.

50. The method of claim 46, wherein said active portion of said polypeptide is encoded by nucleotide coordinates 2025-2079 of SEQ ID NO: 1.

51. The method of claim 47, wherein said active portion of said polypeptide is encoded by nucleotide coordinates 2019-2088 of SEQ ID NO: 1.

52. The method of claim 42, wherein said providing is effected by:
(i) administering said polypeptide to the subject; and/or
(ii) administering an expressible polynucleotide encoding said polypeptide to the subject.

53. The method of claim 42, further comprising providing to the subject a therapeutically effective amount of Tsg101.

54. A nucleic acid construct system comprising:
(a) a first nucleic acid construct including a first polynucleotide encoding at least an active portion of a polypeptide being at least 90% homologous to SEQ ID NO: 2, as determined using the BestFit software of the Wisconsin sequence analysis package, utilizing the Smith and Waterman algorithm, where the gap creation equals 8 and gap extension penalty equals 2; and
(b) a second nucleic acid construct including a second polynucleotide encoding TsglOl or an active portion thereof.

55. The nucleic acid construct system of claim 54, wherein said polypeptide is as set forth in SEQ ID NO: 2, 4 or 6.

56. The nucleic acid construct system of claim 54, wherein said first polynucleotide is at least 85% identical to SEQ ID NO: 1, as determined using the BestFit software of the Wisconsin sequence analysis package, utilizing the Smith and Waterman algorithm, where gap weight equals 50, length weight equals 3, average match equals 10 and average mismatch equals −9.

57. The nucleic acid construct system of claim 54, wherein said first polynucleotide is as set forth in SEQ ID NO: 1, 3 or 5.

58. The nucleic acid construct system of claim 54, wherein said active portion of said polypeptide is as set forth in amino acid coordinates 490-723 of SEQ ID NO: 2.

59. The nucleic acid construct system of claim 54, wherein said active portion of said polypeptide is as set forth in amino acid coordinates 647-723 of SEQ ID NO: 2.

60. The nucleic acid construct system of claim 54, wherein said active portion of said polypeptide is as set forth in amino acid coordinates 647-665 of SEQ ID NO: 2.

61. The nucleic acid construct system of claim 54, wherein said active portion of said polypeptide is as set forth in amino acid coordinates 647-667 of SEQ ID NO: 2.

62. The nucleic acid construct system of claim 58, wherein said active portion of said polypeptide is encoded by nucleotide coordinates 1556-2255 of SEQ ID NO: 1.

63. The nucleic acid construct system of claim 59, wherein said active portion of said polypeptide is encoded by nucleotide coordinates 2025-2255 of SEQ ID NO: 1.

64. The nucleic acid construct system of claim 60, wherein said active portion of said polypeptide is encoded by nucleotide coordinates 2025-2079 of SEQ ID NO: 1.

65. The nucleic acid construct system of claim 61, wherein said active portion of said polypeptide is encoded by nucleotide coordinates 2019-2088 of SEQ ID NO: 1.

66. The nucleic acid construct system of claim 54, wherein each of said first and second nucleic acid constructs further include a promoter for regulating transcription of said first and second polynucleotides in sense or antisense orientation.

67. The nucleic acid construct system of claim 66, wherein said promoter is active in a mammalian cell.

68. A nucleic acid construct comprising a first polynucleotide encoding at least an active portion of a polypeptide being at least 90% homologous to SEQ ID NO: 2, as determined using the BestFit software of the Wisconsin sequence analysis package, utilizing the Smith and Waterman algorithm, where the gap creation equals 8 and gap extension penalty equals 2 and a second polynucleotide encoding Tsg101.

69. The nucleic acid construct of claim 68, wherein said polypeptide is as set forth in SEQ ID NO: 2, 4 or 6.

70. The nucleic acid construct system of claim 36, wherein said first polynucleotide is at least 85% identical to SEQ ID NO: 1, as determined using the BestFit software of the Wisconsin sequence analysis package, utilizing the Smith and Waterman algorithm, where gap weight equals 50, length weight equals 3, average match equals 10 and average mismatch equals −9.

71. The nucleic acid construct of claim 68, wherein said first polynucleotide is as set forth in SEQ ID NO: 1, 3 or 5.

72. The nucleic acid construct of claim 68, wherein said active portion of said polypeptide is as set forth in amino acid coordinates 490-723 of SEQ ID NO: 2.

73. The nucleic acid construct of claim 68, wherein said active portion of said polypeptide is as set forth in amino acid coordinates 647-723 of SEQ ID NO: 2.

74. The nucleic acid construct of claim 68, wherein said active portion of said polypeptide is as set forth in amino acid coordinates 647-667 of SEQ ID NO: 2.

75. The nucleic acid construct of claim 68, wherein said active portion of said polypeptide is as set forth in amino acid coordinates 647-665 of SEQ ID NO: 2.

76. The nucleic acid construct of claim 72, wherein said active portion of said polypeptide is encoded by nucleotide coordinates 1556-2255 of SEQ ID NO: 1.

77. The nucleic acid construct of claim 73, wherein said active portion of said polypeptide is encoded by nucleotide coordinates 2025-2255 of SEQ ID NO: 1.

78. The nucleic acid construct of claim 75, wherein said active portion of said polypeptide is encoded by nucleotide coordinates 2025-2079 of SEQ ID NO: 1.

79. The nucleic acid construct of claim 74, wherein said active portion of said polypeptide is encoded by nucleotide coordinates 2019-2088 of SEQ ID NO: 1.

80. The nucleic acid construct of claim 68, further comprises a promoter for regulating transcription of said first and second polynucleotides in sense or antisense orientation.

81. The nucleic acid construct of claim 80, wherein said promoter is active in a mammalian cell.

82. A method of treating HIV infection and/or a hyperproliferative disease associated with disregulated activity of Tsg101 in a subject, the method comprises downregulating in a subject in need thereof a polypeptide being at least 90% homologous to SEQ ID NO: 2, as determined using the BestFit software of the Wisconsin sequence analysis package, utilizing the Smith and Waterman algorithm, where the gap creation equals 8 and gap extension penalty equals 2, to thereby treat the HIV infection in the subject.

83. The method of claim 82, wherein said downregulating is effected by downregulating a polynucleotide encoding said polypeptide.

84. The method of claim 83, wherein said downregulating said polynucleotide is effected using a ribozyme being specifically hybridizable with said polynucleotide.

85. The method of claim 83, wherein said downregulating said polynucleotide is effected using an antisense being specifically hybridizable with said polynucleotide.

86. The method of claim 83, wherein said downregulating said polynucleotide is effected using a small interfering RNA duplex being specifically hybridizable with said polynucleotide.

87. The method of claim 86, wherein said small interfering RNA duplex is set forth in SEQ ID NOs: 45 and 46.

88. The method of claim 87, wherein said small interfering RNA duplex is set forth in SEQ ID NOs: 47 and 48.

89. The method of claim 83, wherein said downregulating said polypeptide is effected using an antibody.
